(19) United States
(12) Patent Application Publication
(10) Pub. No.: US 2009/0208432 A1
(43) Pub. Date: Aug. 20, 2009

(54) AH Receptor Antagonists

(75) Inventors: Martina Herrmann, Hameln (DE); Gabriele Vielheber, Holzminden (DE); Jean Krutmann, Wegberg (DE); Holger Joppe, Dassel (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co., KG, Holzminden (DE)

(21) Appl. No.: 12/299,214

(22) PCT Filed: Apr. 30, 2007
(86) PCT No.: PCT/EP2007/054205

§ 371 Date: Mar. 27, 2009

Related U.S. Application Data
(60) Provisional application No. 60/796,868, filed on May 3, 2006.

(51) Int. Cl. A61K 31/35; C07D 493/04; A61P 17/16; A61K 8/49

(52) U.S. Cl. 424/59; 549/383; 514/453

(57) ABSTRACT

The invention relates to the field of aryl hydrocarbon receptor (Ah receptor; AhR) antagonists and their uses.

### CROSS-REFERENCE TO RELATED APPLICATIONS

This application claims benefit of priority to PCT/EP2007/054205, filed on Apr. 30, 2007, which asserts priority to U.S. Provisional Application No. 60/796,868, filed on May 3, 2006, which are incorporated herein by reference in their entireties.

The invention relates to the field of aryl hydrocarbon receptor (Ah receptor; AhR) antagonists and their uses.

The skin is the largest organ of the human body. Its most important function is to protect the body on the one hand against the uncontrolled escape of water, and on the other hand against the penetration of harmful chemicals or bacteria, as well as solar radiation.

The exposure of human skin to prolonged solar irradiation can give rise to many kinds of damage. Examples which may be mentioned here are sunburn, photoinduced skin ageing and skin cancer. This harmful action of sunlight is attributed inter alia to the UVB radiation (280-320 nm) contained in the spectrum of sunlight. It is necessary to protect the skin from UVB radiation as comprehensively as possible, particularly in view of the recent large increase in the intensity of the UVB component of the spectrum of sunlight due to the continuing destruction of the ozone layer.

To form a protective layer on the skin in order to protect against UV radiation, conventional sunscreens contain substances which absorb and/or reflect radiation in the 280-400 nm range (UV filters). Examples of such photoprotective substances are inorganic oxides like zinc oxide, or organic UV absorbers like cinnamic acid derivatives or dibenzoylmethane derivatives. However, a disadvantage of these compounds is that the protective layer they form can easily be destroyed by mechanical abrasion, water or detergents. It is therefore desirable to be able to have access not only to said UV filters, but also to substances that develop a protective action inside the skin.

In order to achieve this object, it is of fundamental importance to know the molecular mechanisms by which UVB radiation can cause unhealthy actions on human skin. Appropriate studies have shown that the biological actions of UVB radiation can be attributed in part to the fact that UVB radiation causes structural changes to the DNA molecules in the nucleus of skin cells. Accordingly, DNA repair enzymes are used to provide light protection (Stege et al. (2000) PNAS 97, 1790).

It has also been possible to demonstrate that UVB radiation is capable of triggering changes to the cell membrane that contribute to an activation of growth receptors, such as the epidermal growth factor receptor (EGF-R), and subsequently to tumour formation (Ashida et al. (2003) Exp. Dermatol. 12, 445; Lirvall et al. (1996) Biosci. Rep. 16, 227). This EGF-R activation can be inhibited by antioxidative enzymes (Lirvall et al. (1996) Biosci. Rep. 16, 227).

UVB and UVA light also induces the expression of cyclooxygenase-2 and matrix metalloproteinases (Pentland et al. (1999) Carcinogenesis 20(10), 1939-44). Cyclooxygenases belong to the key enzymes of the inflammatory reaction. They catalyse the first step of the synthesis of a number of inflammation mediators (prostaglandins, prostacyclins, thromboxanes) from arachidonic acid. There are 2 forms: cyclooxygenase-1 (COX-1) is the constitutive, continuously expressed form, whereas COX-2 is only expressed after stimulation by cellular signals, e.g. as a result of tissue damage or inflammation.

Matrix metalloproteinases (MMPs) are enzymes which are capable of proteolytically degrading the macromolecules of the extracellular matrix (ECM). MMPs possess a broad and often overlapping substrate specificity and, in combination, are capable of breaking down all the protein components of the extracellular matrix. About 20 MMPs have so far been identified. MMP-1 (collagenase-1), MMP-2 (gelatinase A), MMP-9 (gelatinase B) and MMP-3, in particular, play an important role in human skin. Apart from collagen-1 and -3, MMP-1 also cleaves Pro-MMP-2 and Pro-MMP-9, thereby activating them. MMP-2 and MMP-9 belong to the elastin-degrading proteases (A. Thibodeau, Cosmetics & Toiletries 2000, 115(11), 75-82).

It has been found that the content of MMPs is markedly greater in old skin than in young skin (J. H. Chung et al., J. Invest. Dermatol. 2001, 117, 1218-1224). MMPs also play a decisive role in the premature skin ageing due to exogenous factors. Thus it has also been possible to detect a higher level of MMPs in skin aged by light than in skin aged with protection from the light (J. H. Chung et al., J. Invest. Dermatol. 2001, 117, 1218-1224). The induction of matrix metalloproteinases has been demonstrated both for UVA and UVB radiation and for infrared radiation. It has been possible to observe this induction both in vitro on cultivated human dermal fibroblasts and in vivo on UV-irradiated human skin. Stimulation with tobacco smoke has also led to an up-regulation of the MMP expression in human dermal fibroblasts.

The aryl hydrocarbon receptor (AhR) (NCBI gene accession number BC0700800) has a central role in the detoxification of exogenous contaminants. It mediates the biological response to polycyclic aromatic hydrocarbons (PAHs) such as benz[a]pyrene and halogenated PAHs such as 2,3,7,8-tetrachlorodibenzo-p-dioxin (TCDD). The AhR is a ligand-activated transcription factor which, after binding a ligand, translocates into the cell nucleus, where it forms a dimer with another transcription factor, namely the aryl hydrocarbon receptor nuclear translocator (ARNT), binds to regulatory gene sequences and induces the transcription of various genes, e.g. CYP1A1 and CYP1B1. The consequences of AhR activation are the development of skin tumours (Shimizu et al. (2000) 97, 779), irritations and inflammations, the development of allergies, atopic dermatitis and itching, and a perturbation of skin integrity (Tauchi et al. (2005) Mol. Cell. Biol. 25, 9360-8; Henley at al., Arch. Biochem. Biophys. (2004) 422, 42-51), as well as the induction of MMP-1 (collagenase-1) (Murphy et al. (2004) J. Biol. Chem. 279, 25284-2593).

UVB light induces CYP1A1 expression in human keratinocytes and lymphocytes and in the mouse hepatoma cell line Hepa-1 (Wei et al., Chem. Biol. Interact. (1999) 118, 127-40). However, it has only been demonstrated for Hepa-1 cells that CYP1A1 induction is AhR-dependent, but, as explained below, AhR activation is dependent on the cell type, so it is not possible to extrapolate from the action on mouse hepatoma cells to the action on human skin cells. Moreover, CYP1A1 can also be induced by AhR-independent pathways (Guigal et al. (2001) Life Sci. 68(18), 2141-50; Tijet et al. (2006) Mol. Pharmacol. 69(1), 140-153). Therefore, there is not necessarily a connection between UVB, the AhR and CYP1A1 in keratinocytes.

WO 99/56737 discloses stilbenes as ligands of the Ah receptor. Although some stilbenes are said to bind to the Ah receptor, none of them effects CYP1A1 induction. These stilbenes include 3,4,3′,5-tetrahydroxystilbene, or piceatannol, 2,3′,4,5′-tetrahydroxystilbene, or oxyresveratrol, and 3,5,4′-trihydroxystilbene, or resveratrol, especially trans-resveratrol. A photoprotective action, particularly against UVB radiation, is not described. A disadvantage of stilbenes is that they are photolabile and frequently elicit endocrine actions. For example, resveratrol is an antiandrogen (Mitchell et al. (1999) Cancer Res. 59, 5892-5895).

Henry et al. ((1999) Mol. Pharmacol. 55, 716-25) describe 3-methoxylated flavones, carrying an electron-withdrawing substituent in the 4-position, as effective AhR antagonists in liver cells. Joiakim et al. ((2003) Drug Metab. Dispos. 31, 1279-82) showed that the Jun N-terminal kinase inhibitor anthra[1,9-cd]pyrazol-6(2H)-one can inhibit the action of the potent AhR agonist 2,3,7,8-tetrachlorodibenzo-p-dioxin (TCDD) in human breast epithelial cells.

Binding to the AhR is furthermore dependent on the cell type. Zhang et al. ((2003) Environ. Health Perspec. 111, 1877-1882) have found that e.g. quercetin prevents the action of the AhR in the human breast cancer cell line MCF-7, but has no effect on the human liver cancer cell line HepG2. An opposite effect was found for luteolin, which has no effect on MCF-7 cells but acts as an AhR inhibitor in HepG2 cells. Differences were also found in the ligand affinity of the AhR between human cells and rodent cells (Ema et al. (1994) J. Biol. Chem. 269, 27337-43; Zhang et al. (2003) Environ. Health Perspec. 111, 1877-1882).

Scarcely any compounds are known which function as AhR antagonists in human skin cells. Although curcumin inhibits AhR activation by the tobacco carcinogen benz[a]pyrene-7R-trans-7,8-dihydrodiol in oral human keratinocyte cancer cells and in ex vivo oral mucosa, it activates AhR translocation in the absence of the tobacco carcinogen (Rinaldi et al. (2002) Cancer Res. 62, 5451-5456) and hence is not an AhR antagonist in terms of the invention.

It is further known that all-trans-retinoic acid inhibits TCDD-induced AhR activation in normal human keratinocytes without influencing AhR activity in the absence of TCDD. All-trans-retinoic acid also has the considerable disadvantage of boosting the TCDD-induced expression of MMP-1 (Murphy et al. (2004) J. Biol. Chem. 279, 25284-25293) and is photolabile.

The object of the invention was therefore to provide substances which act as Ah receptor antagonists without exhibiting the disadvantages of the state of the art as described above.

In terms of this invention, a gene is referred to as induced if the concentration of the corresponding mRNA in the presence of the allocated inductor is significantly higher (p<0.05, Student's t-test), i.e. at least 10% higher, than in the absence of the inductor.

According to the invention, it has now been found that compounds of general

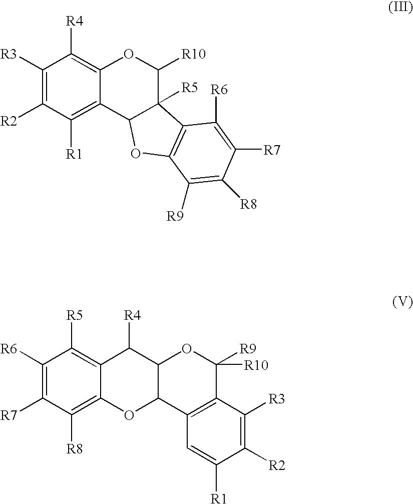

formulae (III) and (V):
in which
R¹ to R¹⁰ independently of one another can be hydrogen, hydroxyl, C₁-C₁₀-alkyl, C₁-C₁₀-alkenyl, C₁-C₁₀-alkoxy, prenyl or O-glycosyl, and two radicals R¹ to R¹⁰ can be joined via a methylenedioxy group —O—CH₂—O—, and
R⁵ can be replaced by a double bond,
are effective as AhR antagonists.

Accordingly, the compounds of formula (III) also include compounds of formula (IV):

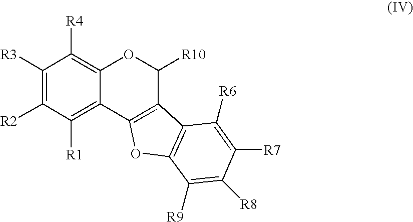

in which the radicals R¹ to R⁴ and R⁶ to R¹⁰ can be the radicals indicated above in respect of formulae (III) and (V).

Surprisingly, the compounds of formulae (III), (IV) and (V) have proved to be very effective Ah receptor antagonists. In human skin cells they can prevent the translocation of the AhR from the cytoplasm into the cell nucleus, especially translocation induced or inducible by UVB and/or by polycyclic aromatic hydrocarbons, particularly TCDD. They greatly reduce the AhR-mediated induction of AhR-inducible genes in human skin cells, especially the induction of CYP1A1. In the absence of an AhR-activating substance, especially AhR inductors such as polycyclic aromatic hydrocarbons and their halogenated derivatives, particularly TCDD, or AhR inductors formed in skin cells (like keratinocytes), such as 6-formylindolo[3,2-b]carbazole (FICZ), they do not trigger the induction of an AhR-inducible gene, nor do they induce the translocation of the AhR from the cytoplasm into the nucleus of human skin cells, in contrast to e.g. curcumin and all-trans-retinoic acid. Presumably by virtue of their influence on the Ah receptor, the compounds of formulae (III), (IV) and (V) are also particularly suitable for reducing or preventing UVB-induced or UVB-inducible skin damage, especially skin cancer, skin ageing, skin inflammations and sunburn.

The compounds of formulae (III), (IV) and (V) are therefore particularly suitable as drugs, especially for treating or preventing (particularly UVB-induced) skin irritations, skin damage, skin inflammations, itching, atopic dermatitis, skin ageing and skin cancer, and/or for reducing the MMP content of the skin. Furthermore, as drugs or in a non-drug form, e.g. as a cosmetic formulation, the compounds are suitable for reducing or preventing a translocation of the AhR into a cell nucleus, reducing or preventing a UVB-induced gene expression, and/or reducing or preventing a gene expression induced by AhR agonists. In this respect the compounds of formulae (III), (IV) and (V) are suitable for protecting skin cells (especially keratinocytes) from environmental toxins, and for detoxification, especially with reference to AhR inductors such as polycyclic aromatic hydrocarbons and their halogenated derivatives, particularly TCDD, or AhR inductors formed in skin cells, such as 6-formylindolo[3,2-b]carbazole (FICZ).

The compounds of formulae (III), (IV) and (V) are also suitable as sunscreens and especially as UVB filters.

Surprisingly, the compounds of formulae (III), (IV) and (V) additionally have a skin-lightening action.

According to the invention, the compounds of formulae (III), (IV) and (V) are used as drugs, as constituents of a pharmaceutical formulation or as constituents of a non-pharmaceutical formulation, especially a cosmetic formulation. Where reference is made hereafter to “preferred substances according to the invention” and their properties and possible uses, this always includes the corresponding drug or the corresponding pharmaceutical or non-pharmaceutical formulation, unless indicated otherwise.

Preferred compounds of formulae (III), (IV) and (V) according to the invention are those in which no more than 4 of the radicals R¹ to R⁴ and R⁶ to R¹⁰ are not hydrogen. Other preferred compounds of formulae (III), (IV) and (V) are those in which the radicals R¹ to R¹⁰ independently of one another are hydrogen, hydroxyl, C₁-C₂-alkyl or C₁-C₂-alkoxy; preferably, no more than 4 of the radicals R¹ to R⁴ and R⁶ to R¹⁰ are as defined above and are not hydrogen. Compounds substituted in this way have proved to be particularly effective AhR antagonists. Particularly preferred compounds are those of the formulae

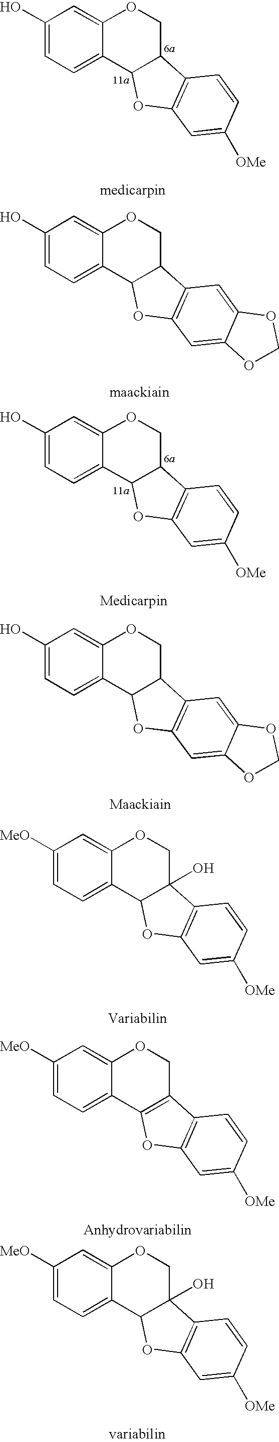

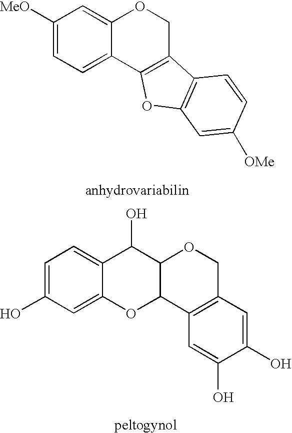

The compounds afford a strong inhibition of the Ah receptor, even in very low use concentrations, and prevent or reduce the AhR-mediated induction of AhR-inducible genes, especially CYP1A1, even in low concentrations. Furthermore, in the absence of UVB radiation and/or an AhR inductor, preferably TCDD, the compounds of the do not themselves induce an AhR-inducible gene, especially CYP1A1, nor do they induce the translocation of the AhR from the cytoplasm into the cell nucleus under these conditions. The compounds are therefore particularly suitable for the above-described uses of AhR antagonists and are especially preferred as drugs.

The formulations according to the invention contain the compound(s) of formulae (III), (IV) and/or (V) in a concentration preferably of at least 0.0001 wt. %, based on the total composition. In these concentrations, especially in the case of the specific compound mentioned, it is already possible to observe a reduction in the translocation of the AhR receptor into the nucleus of skin cells, and also the induction of AhR-inducible genes, especially CYP1A1, for example by TCDD, is already significantly reduced.

Preferably, the concentration of the compound(s) of formulae (III), (IV) and (V) is 0.0005 to 15 wt. %, particularly preferably 0.001 to 10 wt. % and especially 0.01 to 5 wt. %, based in each case on the total weight of the composition. When applied to the skin in these concentrations, the compounds of formulae (III), (IV) and (V) develop a strong AhR-antagonistic action in that they prevent or reduce the translocation of the AhR into the cell nucleus and, in particular, reduce or prevent a UVB-induced gene expression, specifically of CYP1A1.

Cosmetic or pharmaceutical formulations that consist essentially of one or more compounds of formulae (III), (IV) and/or (V) contain these compounds in a proportion of at least 90 wt. % and preferably of at least 35 wt. %, based on the total formulation.

The formulations can be especially cosmetic formulations, particular preference being afforded to sun creams, skin protection lotions and after-sun lotions.

The cosmetic or therapeutic formulations according to the invention are prepared by conventional processes known per se, wherein the compound(s) of formulae (III), (IV) and (V) is (are) incorporated into cosmetic or dermatological formulations which are of conventional composition and, in addition to their detoxifying and/or skin-lightening and hair-lightening action, can also be used to treat, care for and clean the skin or hair.

The formulations according to the invention preferably take the form of an emulsion, e.g. an emulsion of the W/O (water-in-oil) type, O/W (oil-in-water) type, W/O/W (water-in-oil-in-water) type or O/W/O (oil-in-water-in-oil) type, a PIT emulsion, a Pickering emulsion, an emulsion with a low oil content, or a microemulsion or nanoemulsion, a solution, e.g. in oil (fatty oils or fatty acid esters, especially C₆-C₃₂ fatty acid C₂-C₃₀ esters) or silicone oil, a dispersion, a suspension, a cream, a lotion or milk, depending on the preparative process and the ingredients, a gel (including hydrogel, hydrodispersion gel, oleogel), a spray (e.g. pump spray or spray with propellant) or else a foam or an impregnating solution for cosmetic tissues, a cleaning product, e.g. soap, syndet, liquid wash, shower or bath preparation, a bath product (capsule, oil, tablet, salt, bath salt, soap, etc.), an effervescent formulation, a skin care product such as an emulsion (as described above), an ointment, a paste, a gel (as described above), an oil, a toner, a balsam, a serum or a powder (e.g. face powder, body powder), a mask, a pencil, a stick, a roll-on, a pump, an aerosol (foaming, non-foaming or after-foaming), a deodorant and/or antiperspirant, a gargle or mouthwash, a foot care product (including keratolytic, deodorant), an insect repellent, a sunscreen, a self-tanning product and/or after-sun preparation, a skin toner, a shaving product, an after-shave balm, a pre-shave or after-shave lotion, a depilatory, a hair care product, e.g. a shampoo (including 2-in-1 shampoo, antidandruff shampoo, baby shampoo, shampoo for dry scalp, shampoo concentrate), a conditioner, a hair treatment, a hair tonic, a hair rinse, a styling cream, a pommade, a perming product or fixer, a hair setting lotion (spray), a styling aid (e.g. gel or wax), a hair smoothing product (straightener, relaxer), a bleach, a hair colourant, e.g. temporary, direct hair colourant, semipermanent hair colourant or permanent hair colourant, a hair toner, a hair lightener, a hair conditioner, a hair mousse, an eye care product, a make-up, a make-up remover or a baby product.

It is also advantageous to administer the compound(s) of formulae (III), (IV) and (v) in encapsulated form, e.g. in gelatin, wax materials, liposomes or cellulose capsules.

Particularly preferably, the formulations according to the invention take the form of an emulsion, especially an emulsion of the W/O, O/W, W/O/W or O/W/O type, a PIT emulsion, a Pickering emulsion, an emulsion with a low oil content, or a microemulsion or nanoemulsion, a gel (including hydrogel, hydrodispersion gel, oleogel), a solution, e.g. in oil (fatty oils or fatty acid esters, especially C₆-C₃₂ fatty acid C₂-C₃₀ esters) or silicone oil, or a spray (e.g. pump spray or spray with propellant).

The (particularly topical) cosmetic or therapeutic formulations according to the invention can preferably contain cosmetic and/or dermatological auxiliary substances and additives such as those conventionally used in such formulations, e.g. cooling agents, sunscreens (especially UV filters and/or UV-filtering pigments), dyes, pigments with a colouring action, antioxidants, preservatives, anti-irritants, softeners, moisturizers and/or moisture retainers (moisture retention regulators, e.g. glycerol or urea), osmolytes, antimicrobials (e.g. antibacterials, bactericides, fungicides), virucides, deodorants (e.g. antiperspirants), surface-active substances (surfactants), emulsifiers, insect repellents (e.g. DEET, IR 3225, Dragorepel), plant extracts, anti-inflammatories, cicatrizants (e.g. chitin or chitosan and chitosan derivatives), gelling agents, film-forming substances (e.g. polyvinylpyrrolidones or chitosan or chitosan derivatives), fixatives, skin smoothing substances, antiwrinkle substances such as beta-glucan from oats, blackberry leaf extract or soya extract, vitamins (e.g. vitamin C and derivatives, tocopherols and derivatives, vitamin A and derivatives), 2-hydroxycarboxylic acids (e.g. citric acid, malic acid, L-, D- or DL-lactic acid), skin colourants (e.g. walnut extracts or dihydroxyacetone), skin care and repair agents (e.g. cholesterol, ceramides, pseudoceramides, creatine and creatine esters), skin soothing agents, superfatting agents, optical brighteners, lubricants, lustre agents, fats, oils, saturated fatty acids and salts thereof, monounsaturated or polyunsaturated fatty acids and salts thereof, alpha-hydroxy acids, polyhydroxy fatty acids or derivatives thereof (e.g. linoleic acid, alpha-linolenic acid, gamma-linolenic acid or arachidonic acid and natural or synthetic esters thereof), phospholipids, waxes or other conventional constituents of a cosmetic or dermatological formulation, such as alcohols, alkanediols, polyols, polymers, electrolytes, organic solvents, silicones, silicone derivatives or chelating agents (e.g. ethylenediaminetetraacetic acid and derivatives), antidandruff agents (e.g. climbazole, ketoconazole, piroctonoleamine, zinc pyrithione), hair care agents, hair deforming agents, hair smoothing agents, depilatories, perfumes, ethereal oils, foaming agents, foam stabilizers, foam boosters, antifoams, thickeners, binders, plant parts (e.g. fibres) and plant extracts (e.g. arnica, aloe, beard lichen, ivy, stinging nettle, ginseng, henna, camomile, marigold, rosemary, sage, horsetail or thyme), animal extracts, e.g. royal jelly, propolis, proteins or protein hydrolysates, yeast extracts, hop and wheat extracts, peptides or thymus extracts, abrasives, buffers and enzymes.

Constituents (auxiliary substances and additives) with which the compound(s) of formulae (III), (IV) and (V) can be combined are particularly preferred:

abrasives, antidandruff agents, anti-inflammatories, antioxidants, antiperspirants, binders, buffers, chelating agents, depilatories, surface-active substances, emulsifiers, enzymes, ethereal oils, plant extracts, fibres, film-forming agents, fixatives, foaming agents, foam stabilizers, antifoams, foam boosters, gelling agents, hair care agents, hair deforming agents, hair smoothing agents, skin and hair lighteners, moisturizers, moisture retainers, insect repellents, optical brighteners, lubricants, lustre agents, polymers, proteins, superfatting agents, skin soothing agents, skin smoothing agents, antiwrinkle agents, sunscreens, vitamins, oils, waxes, fats, phospholipids, saturated fatty acids and salts thereof, monounsaturated or polyunsaturated fatty acids and salts thereof, alpha-hydroxy acids, polyhydroxy fatty acids, polyols, alkanediols, silicones or silicone derivatives.

Auxiliary substances and additives can be present in amounts of 5 to 99 wt. %, preferably of 10 to 80 wt. %, based on the total weight of the formulation. The amounts of cosmetic or dermatological auxiliary substances and additives and perfume to be used in each case can easily be determined by those skilled in the art, according to the particular type of product, using simple trial and error.

The formulations can also contain water in an amount of up to 99.99 wt. %, preferably of 5 to 80 wt. %, based on the total weight of the formulation.

Cosmetic or therapeutic formulations according to the invention are preferably formulations which are selected from the group comprising:

an emulsion, a solution, a dispersion, a suspension, a cream, a lotion, a milk, a gel, a spray, a foam, an impregnating solution for cosmetic tissues, a cleaning product, a soap, a syndet, a wash preparation, a shower preparation, a bath preparation, a bath product, an effervescent formulation, a skin care product, an ointment, a paste, an oil, a toner, a balsam, a serum, a powder, a mask, a pencil, a stick, a roll-on, a pump, an aerosol, a deodorant, an antiperspirant, a gargle, a mouthwash, a foot care product, an insect repellent, a sunscreen, a self-tanning product, an after-sun preparation, a skin toner, a shaving product, an after-shave balm, a pre-shave lotion, an after-shave lotion, a depilatory, a hair care product, a shampoo, a conditioner, a hair treatment, a hair tonic, a hair rinse, a styling cream, a pommade, a perming product, a fixer, a hair setting lotion, a styling aid, a hair smoothing product, a bleach, a hair colourant, a hair toner, a hair lightener, a hair conditioner, a hair mousse, an eye care product, a make-up, a make-up remover and a baby product,
and/or
which, in addition to the compound(s) of formulae (III), (IV) and (V), contain one or more auxiliary substances and additives selected from the group comprising:
abrasives, antidandruff agents, anti-inflammatories, antioxidants, antiperspirants, binders, buffers, chelating agents, depilatories, surface-active substances, emulsifiers, enzymes, ethereal oils, plant extracts, fibres, film-forming agents, fixatives, foaming agents, foam stabilizers, antifoams, foam boosters, gelling agents, hair care agents, hair deforming agents, hair smoothing agents, skin and hair lighteners, moisturizers, moisture retainers, insect repellents, optical brighteners, lubricants, lustre agents, polymers, proteins, superfatting agents, skin soothing agents, skin smoothing agents, antiwrinkle agents, sunscreens, vitamins, oils, waxes, fats, phospholipids, saturated fatty acids and salts thereof, monounsaturated or polyunsaturated fatty acids and salts thereof, alpha-hydroxy acids, polyhydroxy fatty acids, polyols, alkanediols, silicones and silicone derivatives,
and/or
which are intended for application to the hair and/or skin.

For use, the formulations containing the compound(s) of formulae (III), (IV) and (v) are normally applied to the skin and/or hair in a sufficient amount and in a manner customary for cosmetic and skin preparations. Cosmetic, dermatological and/or therapeutic formulations according to the invention which additionally comprise one or more sunscreens (UV absorbers, UV filters) are particularly advantageous.

In rare cases, formulations and drugs containing compounds formulae (III), (IV) and (V) according to the invention, or to be used according to the invention, can experience discolouration and/or instability, especially if they are in aqueous-alcoholic or purely alcoholic solution. Surprisingly, it has now been found that UV filters can improve the stability of the compounds of formulae (III), (IV) and (V) in formulations and drugs according to the invention. In particular, UV filters can prevent or retard a discolouration of the compounds of formulae (III), (IV) and (V) due to sunlight or other light. Both are important, especially in cosmetic formulations. According to the invention, UV filters are therefore used to stabilize the compounds of formulae (III), (IV) and (V), especially by using one or more UV filters in a sufficient amount to stabilize the compounds of formulae (III), (IV) and (V) in a formulation according to the invention, and preferably using the UV filters mentioned below (as preferred). For stabilization purposes, the total amount of UV filters ranges preferably from 0.1 to 2 wt. % and particularly preferably from 0.2 to 1 wt. %, based on the total weight of the formulation.

In this context a further feature of the invention relates to the cosmetic or therapeutic use of one or more compounds of formulae (III), (IV) and (V) for lightening the skin and/or hair in the presence of one or more UV filters in an amount that stabilizes the compound(s) of formulae (III), (IV) and (V), all the details given above on the choice of substituents naturally applying in this case as well.

The ratio of the total proportion by weight of UV filters to the total proportion by weight of compounds of formulae (III), (IV) and (V) according to the invention, or to be used according to the invention, ranges preferably from 100:1 to 1:100, particularly preferably from 10:1 to 1:10 and very particularly preferably from 5:1 to 1:5.

Formulations according to the invention which contain one or more UV filters (sunscreens, UV absorbers) have a total proportion of UV filters ranging preferably from 0.1 to 30 wt. %, particularly preferably from 0.2 to 20 wt. % and very particularly preferably from 0.5 to 15 wt. %, based on the total weight of the formulation. Particularly preferably, the formulations according to the invention contain one or more UVB filters, especially of the types indicated below. Surprisingly, it has been found that the substances of formulae (III), (IV) and (V) according to the invention interact advantageously with UVB filters to prevent UVB-induced skin damage, skin changes and skin cancer, and that, in particular, formulations and drugs containing compounds of formulae (III), (IV) and (V) together with UVB filters remain effective for a particularly long time.

Particularly preferably, the compounds of formulae (III), (IV) and (V) according to the invention, or to be used according to the invention, are combined with water-soluble UV filters; in one preferred embodiment, they are combined with disodium phenylenebisbenzimidazyltetrasulfonate (Neo Heliopan® AP) and/or 2-phenylbenzimidazolesulfonic acid (Neo Heliopan® Hydro).

In another preferred embodiment, a formulation according to the invention contains sunscreens, i.e. especially UV filters and/or inorganic pigments (UV-filtering pigments), in a total amount such that the formulation according to the invention has a light protection factor greater than or equal to 2 (preferably greater than or equal to 5 and particularly preferably greater than or equal to 10). Such formulations according to the invention are particularly suitable for protecting the skin and hair.

Formulations according to the invention which additionally comprise one or more sunscreens (UV filters, UV absorbers) can take a variety of forms such as those conventionally used e.g. for sunscreen formulations. For example, they can be in the form of an emulsion of the oil-in-water (O/W) type, a gel, a hydrodispersion or an aerosol.

Advantageously, the formulations according to the invention contain at least one UVA filter and/or at least one UVB filter and/or a broadband filter and/or at least one inorganic pigment. Formulations according to the invention preferably contain at least one UVB filter or a broadband filter and particularly preferably contain at least one UVA filter and at least one UVB filter.

Examples of suitable UV filters are organic UV absorbers from the class comprising 4-aminobenzoic acid and derivatives, salicylic acid derivatives, benzophenone derivatives, dibenzoylmethane derivatives, diphenyl acrylates, 3-imidazol-4-ylacrylic acid and esters thereof, benzofuran derivatives, benzylidenemalonate derivatives, polymeric UV absorbers containing one or more silicon-organic radicals, cinnamic acid derivatives, camphor derivatives, trianilino-s-triazine derivatives, 2-hydroxyphenylbenzotriazole derivatives, phenylbenzimidazolesulfonic acid derivatives and salts thereof, menthyl anthranilate, benzotriazole derivatives and indole derivatives.

The UV filters mentioned below, which can be used for the purposes of the present invention, are preferred but of course do not imply a limitation.

Advantageous UV filters are:

UVB filters such as:
p-aminobenzoic acid
ethoxylated ethyl p-aminobenzoate (25 mol of EO) (INCI name: PEG-25 PABA)
2-ethylhexyl p-dimethylaminobenzoate
N-propoxylated ethyl p-aminobenzoate (2 mol of PO)
glyceryl p-aminobenzoate
homomethyl salicylate (homosalate) (Neo Heliopan® HMS)
2-ethylhexyl salicylate (Neo Heliopan® OS)
triethanolamine salicylate
4-isopropylbenzyl salicylate
menthyl anthranilate (Neo Heliopan® MA)
ethyl diisopropylcinnamate
2-ethylhexyl p-methoxycinnamate (Neo Heliopan® AV) methyl diisopropylcinnamate
isoamyl p-methoxycinnamate (Neo Heliopan® E 1000)
diethanolamine p-methoxycinnamate
isopropyl p-methoxycinnamate
2-phenylbenzimidazolesulfonic acid and salts (Neo Heliopan® Hydro)
3-(4′-trimethylammonium)benzylidenebornan-2-one methylsulfate
beta-imidazol-4(5)-acrylic acid (urocanic acid)
3-(4′-sulfo)benzylidenebornan-2-one and salts
3-(4′-methylbenzylidene)-D,L-camphor (Neo Heliopan® MBC)
3-benzylidene-D,L-camphor
N-[(2 and 4)-[2-(oxoborn-3-ylidene)methyl]benzyl]acrylamide polymer
4,4′-[(6-[4-(1,1-dimethylaminocarbonyl)phenylamino]-1,3,5-triazine-2,4-diyl)diimino]bis(2-ethylhexyl benzoate) (Uvasorb® HEB)
benzylidenemalonate-polysiloxane (Parsol® SLX)
glyceryl ethylhexanoate dimethoxycinnamate
dipropylene glycol salicylate
tris(2-ethylhexyl) 4,4′,4″-(1,3,5-triazine-2,4,6-triyltriimino)tribenzoate (=2,4,6-trianilino(p-carbo-2′-ethylhexyl-1′-oxy)-1,3,5-triazine) (Uvinul® T150)
   broadband filters such as:
2-ethylhexyl 2-cyano-3,3-diphenylacrylate (Neo Heliopan® 303)
ethyl 2-cyano-3,3′-diphenylacrylate
2-hydroxy-4-methoxybenzophenone (Neo Heliopan® BB)
2-hydroxy-4-methoxybenzophenone-5-sulfonic acid
dihydroxy-4-methoxybenzophenone
2,4-dihydroxybenzophenone
tetrahydroxybenzophenone
2,2′-dihydroxy-4,4′-dimethoxybenzophenone
2-hydroxy-4-n-octyloxybenzophenone
2-hydroxy-4-methoxy-4′-methylbenzophenone
sodium hydroxymethoxybenzophenonesulfonate
disodium 2,2′-dihydroxy-4,4′-dimethoxy-5,5′-disulfo-benzophenone
2-(2H-benzotriazol-2-yl)-4-methyl-6-(2-methyl-3-(1,3,3,3-tetramethyl-1-(trimethylsilyl)oxy)disiloxanyl)propylphenol (Mexoryl® XL)
2,2′-methylenebis(6-(2H-benztriazol-2-yl)-4-(1,1,3,3-tetramethylbutyl)phenol) (Tinosorb® M)
2,4-bis[4-(2-ethylhexyloxy)-2-hydroxyphenyl]-1,3,5-triazine
2,4-bis[{4-(2-ethylhexyloxy)-2-hydroxy}phenyl]-6-(4-methoxyphenyl)-1,3,5-triazine (Tinosorb® S)
2,4-bis[{(4-(3-sulfonato)-2-hydroxypropoxy)-2-hydroxy}phenyl]-6-(4-methoxyphenyl)-1,3,5-triazine sodium salt
2,4-bis[{(3-(2-propoxy)-2-hydroxypropoxy)-2-hydroxy}phenyl]-6-(4-methoxyphenyl)-1,3,5-triazine
2,4-bis[{4-(2-ethylhexyloxy)-2-hydroxy}phenyl]-6-[4-(2-methoxyethylcarbonyl)phenylamino]-1,3,5-triazine
2,4-bis[{4-(3-(2-propoxy)-2-hydroxypropoxy)-2-hydroxy}phenyl]-6-[4-(2-ethylcarboxyl)phenylamino]-1,3,5-triazine
2,4-bis[{4-(2-ethylhexyloxy)-2-hydroxy}phenyl]-6-(1-methylpyrrol-2-yl)-1,3,5-triazine
2,4-bis[{4-tris(trimethylsiloxysilylpropoxy)-2-hydroxy}phenyl]-6-(4-methoxyphenyl)-1,3,5-triazine
2,4-bis[{4-(2″-methylpropenyloxy)-2-hydroxy}phenyl]-6-(4-methoxyphenyl)-1,3,5-triazine
2,4-bis[{4-(1′,1′,1′,3′,5′,5′,5′-heptamethylsiloxy-2″-methylpropoxy)-2-hydroxy}phenyl]-6-(4-methoxyphenyl)-1,3,5-triazine
   UVA filters such as:
4-isopropyldibenzoylmethane
terephthalylidenedibornanesulfonic acid and salts (Mexoryl® SX)
4-t-butyl-4′-methoxydibenzoylmethane (avobenzone) (Neo Heliopan® 357)
phenylenebisbenzimidazyltetrasulfonic acid disodium salt (Neo Heliopan® AP)
2,2′-(1,4-phenylene)bis(1H-benzimidazole-4,6-disulfonic acid) monosodium salt
hexyl 2-(4-diethylamino-2-hydroxybenzoyl)benzoate (Uvinul® A Plus)
indanylidene compounds according to DE 100 55 940
   (=WO 02/38537)
      The following UV filters are particularly suitable for combination:
p-aminobenzoic acid
3-(4′-trimethylammonium)benzylidenebornan-2-one methylsulfate
homomethyl salicylate (Neo Heliopan® HMS)
2-hydroxy-4-methoxybenzophenone (Neo Heliopan® BB)
2-phenylbenzimidazolesulfonic acid (Neo Heliopan® Hydro)
terephthalylidenedibornanesulfonic acid and salts (Mexoryl® SX)
4-tert-butyl-4′-methoxydibenzoylmethane (Neo Heliopan® 357)
3-(4′-sulfo)benzylidenebornan-2-one and salts
2-ethylhexyl 2-cyano-3,3-diphenylacrylate (Neo Heliopan®303)
N-[(2 and 4)-[2-(oxoborn-3-ylidene)methyl]benzyl]acrylamide polymer
2-ethylhexyl p-methoxycinnamate (Neo Heliopan® AV)
ethoxylated ethyl p-aminobenzoate (25 mol of EO) (INCI name: PEG-25 PABA)
isoamyl p-methoxycinnamate (Neo Heliopan® E1000)
2,4,6-trianilino(p-carbo-2′-ethylhexyl-1′-oxy)-1,3,5-triazine (Uvinul® T150)
2-(2H-benzotriazol-2-yl)-4-methyl-6-(2-methyl-3-(1,3,3,3-tetramethyl-1-(trimethylsilyl)oxy)disiloxanyl)propylphenol (Mexoryl® XL)
4,4′-[(6-[4-(1,1-dimethyl)aminocarbonyl)phenylamino]-1,3,5-triazine-2,4-diyl)diimino]bis(2-ethylhexyl benzoate) (Uvasorb® HEB)
3-(4′-methylbenzylidene)-D,L-camphor (Neo Heliopan® MBC)
3-benzylidenecamphor
2-ethylhexyl salicylate (Neo Heliopan® OS)
2-ethylhexyl 4-dimethylaminobenzoate (Padimate O)
hydroxy-4-methoxybenzophenone-5-sulfonic acid and Na salt
2,2′-methylenebis(6-(2H-benztriazol-2-yl)-4-(1,1,3,3-tetramethylbutyl)phenol) (Tinosorb® M)
phenylenebisbenzimidazyltetrasulfonic acid disodium salt (Neo Heliopan® AP)
2,4-bis[{4-(2-ethylhexyloxy)-2-hydroxy}phenyl]-6-(4-methoxyphenyl)-1,3,5-triazine (Tinosorb® S)
benzylidenemalonate-polysiloxane (Parsol® SLX)
menthyl anthranilate (Neo Heliopan® MA)
hexyl 2-(4-diethylamino-2-hydroxybenzoyl)benzoate (Uvinul® A Plus)
indanylidene compounds according to DE 100 55 940
   (=WO 02/38537)

It is also possible to use particulate UV filters or inorganic pigments, which can optionally be hydrophobized, such as the oxides of titanium (TiO₂), zinc (ZnO), iron (Fe₂O₃), zirconium (ZrO₂), silicon (SiO₂), manganese (e.g. MnO), aluminium (Al₂O₃) and cerium (e.g. Ce₂O₃) and/or mixtures thereof.

Formulations according to the invention, especially dermatological formulations, can also advantageously contain dyes and/or coloured pigments, particularly if they are to be used in the field of decorative cosmetics. The dyes and coloured pigments can be selected from the appropriate list approved by the cosmetics regulations or from the EC list of cosmetic colourants. In most cases they are identical to the dyes permitted for use in foods. Examples of advantageous coloured pigments are titanium dioxide, mica, iron oxides (e.g. Fe₂O₃, Fe₃O₄, FeO(OH)) and/or tin oxide. Examples of advantageous dyes are carmine, Berlin blue, chrome oxide green, ultramarine blue and/or manganese violet.

Specific cooling agents preferably used within the framework of the present invention are listed below. Those skilled in the art can add a large number of other cooling agents to this list; the cooling agents listed can also be used in combination with one another: L-menthol, D-menthol, racemic menthol, menthone glycerol acetal (trade name: Frescolat® MGA), menthyl lactate (trade name: Frescolat® ML; menthyl lactate is preferably L-menthyl lactate, especially L-menthyl L-lactate), substituted menthyl-3-carboxamides (e.g. menthyl-3-carboxylic acid N-ethylamide), 2-isopropyl-N-2,3-trimethylbutanamide, substituted cyclohexanecarboxamides, 3-menthoxypropane-1,2-diol, 2-hydroxyethyl menthyl carbonate, 2-hydroxypropyl menthyl carbonate, N-acetylglycine menthyl ester, isopulegol, hydroxycarboxylic acid menthyl esters (e.g. menthyl 3-hydroxybutyrate), monomenthyl succinate, 2-mercaptocyclodecanone, menthyl 2-pyrrolidin-5-onecarboxylate, 2,3-dihydroxy-p-menthane, 3,3,5-trimethylcyclohexanone glycerol ketal, 3-menthyl-3,6-di- and -trioxaalkanoates, 3-menthyl methoxyacetate and icilin.

Preferred cooling agents are L-menthol, D-menthol, racemic menthol, menthone glycerol acetal (trade name: Frescolat® MGA), menthyl lactate (preferably L-menthyl lactate, especially L-menthyl L-lactate (trade name: Frescolat® ML)), substituted menthyl-3-carboxamides (e.g. menthyl-3-carboxylic acid N-ethylamide), 2-isopropyl-N-2,3-trimethylbutanamide, substituted cyclohexanecarboxamides, 3-menthoxypropane-1,2-diol, 2-hydroxyethyl menthyl carbonate, 2-hydroxypropyl menthyl carbonate and isopulegol.

Particularly preferred cooling agents are L-menthol, racemic menthol, menthone glycerol acetal (trade name: Frescolat® MGA), menthyl lactate (preferably L-menthyl lactate, especially L-menthyl L-lactate (trade name: Frescolat® ML)), 3-menthoxypropane-1,2-diol, 2-hydroxyethyl menthyl carbonate and 2-hydroxy-propyl menthyl carbonate.

Very particularly preferred cooling agents are L-menthol, menthone glycerol acetal (trade name: Frescolat® MGA) and menthyl lactate (preferably L-menthyl lactate, especially L-menthyl L-lactate (trade name: Frescolat® ML)).

Depending on the substance, the use concentration of the cooling agents to be used ranges preferably from 0.01 to 20 wt. % and particularly preferably from 0.1 to 5 wt. %, based on the total weight of the finished (ready-to-use), preferably topical, cosmetic or therapeutic (pharmaceutical) formulation.

The formulations according to the invention can preferably contain other skin and hair lightening compounds suitable for cosmetic (e.g. dermatological) and/or therapeutic applications. Advantageous skin and hair lightening compounds in this context are kojic acid (5-hydroxy-2-hydroxymethyl-4-pyranone), kojic acid derivatives, e.g. kojic acid dipalmitate, arbutin, ascorbic acid, ascorbic acid derivatives, hydroquinone, hydroquinone derivatives, resorcinol, sulfur-containing molecules, e.g. glutathione or cysteine, alpha-hydroxy acids (e.g. citric acid, lactic acid, malic acid) and derivatives thereof, N-acetyltyrosine and derivatives, undecenoylphenylalanine, gluconic acid, 4-alkylresorcinols, 4-(1-phenylethyl)-1,3-dihydroxybenzene, chromone derivatives such as aloesin, flavonoids, thymol derivatives, 1-aminoethylphosphinic acid, thiourea derivatives, ellagic acid, nicotinamide (niacinamide), zinc salts, e.g. zinc chloride or gluconate, thujaplicin and derivatives, triterpenes such as maslinic acid, sterols such as ergosterol, benzofuranones such as senkyunolide, vinyl- and ethylguaiacol, dioic acids such as octadecenedioic acid and azelaic acid, inhibitors of nitrogen oxide synthesis, e.g. L-nitroarginine and derivatives thereof, 2,7-dinitroindazole or thiocitrullin, metal chelators (e.g. alpha-hydroxy fatty acids, palmitic acid, phytic acid, lactoferrin, humic acid, gallic acid, bile extracts, bilirubin, biliverdin, EDTA, EGTA and derivatives thereof), retinoids, soya milk and soya extract, serine protease inhibitors or lipoic acid, or other synthetic or natural skin and hair lightening compounds, the latter also being used in the form of a plant extract, e.g. bearberry extract, rice extract, papaya extract, licorice extract or components obtained therefrom by enrichment, such as glabridin or licochalcone A, Artocarpus extract, extracts of Rumex and Ramulus species, extracts of pine species (Pinus) and extracts of Vitis species, or stilbene derivatives obtained therefrom by enrichment, and Saxifraga, mulberry, Scutelleria and/or grape extracts.

The amount of the aforementioned exemplary other skin and hair lightening compounds (one or more compounds) in the formulations according to the invention is then preferably 0.005 to 30 wt. %, particularly preferably 0.01 to 20 wt. % and very particularly preferably 0.01 to 5 wt. %, based on the total weight of the formulation.

As dyes formulations according to the invention (especially if application to the facial area is intended), it can be advantageous to choose one or more substances from the following group: 2,4-dihydroxyazobenzene, 1-(2′-chloro-4′-nitro-1′-phenylazo)-2-hydroxynaphthalene, ceres red, 2-(4-sulfo-1-naphthylazo)-1-naphthol-4-sulfonic acid, calcium 2-hydroxy-1,2′-azonaphthalene-1′-sulfonate, calcium and barium salts of 1-(2-sulfo-4-methyl-1-phenylazo)-2-naphthylcarboxylic acid, the calcium salt of 1-(2-sulfo-1-naphthylazo)-2-hydroxynaphthalene-3-carboxylic acid, the aluminium salt of 1-(4-sulfo-1-phenylazo)-2-naphthol-6-sulfonic acid, the aluminium salt of 1-(4-sulfo-1-naphthylazo)-2-naphthol-3,6-disulfonic acid, 1-(4-sulfo-1-naphthylazo)-2-naphthol-6,8-disulfonic acid, the aluminium salt of 4-(4-sulfo-1-phenylazo)-1-(4-sulfophenyl)-5-hydroxypyrazolone-3-carboxylic acid, aluminium and zirconium salts of 4,5-dibromofluorescein, aluminium and zirconium salts of 2,4,5,7-tetrabromofluorescein, 3′,4′,5′,6′-tetrachloro-2,4,5,7-tetrabromofluorescein and its aluminium salt, the aluminium salt of 2,4,5,7-tetraiodofluorescein, the aluminium salt of quinophthalonedisulfonic acid, the aluminium salt of indigodisulfonic acid, red and black iron oxide (Colour Index Number (CIN): 77491 (red) and 77499 (black)), hydrated iron oxide (CIN: 77492), manganese ammonium diphosphate and titanium dioxide.

Other advantageous dyes are oil-soluble natural dyes such as paprika extracts, β-carotene or cochineal.

Dermatological formulations containing pearlescent pigments are also advantageous for the purposes of the present invention. The types of pearlescent pigments listed below are particularly preferred:

1. natural pearlescent pigments such as:
pearl essence (guanine/hypoxanthine mixed crystals from fish scales) and
mother of pearl (ground mussel shells)
   2. monocrystalline pearlescent pigments such as bismuth oxychloride (BiOCl)
   3. sheet pigments, e.g. mica/metal oxide

Pearlescent pigments are based e.g. on pulverulent pigments or castor oil dispersions of bismuth oxychloride and/or titanium dioxide and bismuth oxychloride and/or titanium dioxide on mica. The lustre pigment listed under CIN 77163, for example, is particularly advantageous.

Of course, the stated list of pearlescent pigments shall not imply a limitation. Pearlescent pigments which are advantageous for the purposes of the present invention are obtainable by a large number of methods known per se. For example, substrates other than mica, e.g. silica and the like, can also be coated with other metal oxides. For example, SiO₂ particles coated with TiO₂ and Fe₂O₃ (“Ronaspheres”), which are marketed by Merck and are particularly suitable for the optical reduction of fine wrinkles, are advantageous.

Furthermore, it may be advantageous to dispense completely with a substrate like mica. Particular preference is afforded to iron pearlescent pigments which are prepared without using mica. Such pigments are obtainable e.g. under the trade name Sicopearl Kupfer 1000 from BASF.

Effect pigments obtainable in different colours (yellow, red, green, blue) from Flora Tech under the trade name Metasomes Standard/Glitter are also particularly advantageous. The Glitter particles here take the form of mixtures with various auxiliary substances and dyes (e.g. the dyes of CIN 19140, 77007, 77289, 77491).

The dyes and pigments can be present either individually or in a mixture and can be coated with one another, different colour effects generally being created by different coating thicknesses. The total amount of dyes and coloured pigments is advantageously chosen within the range from e.g. 0.1 wt. % to 30 wt. %, preferably from 0.5 to 15 wt. % and particularly preferably from 1.0 to 10 wt. %, based in each case on the total weight of the (cosmetic) formulations.

The formulations according to the invention can also contain (additional) antioxidants or preservatives. Any antioxidants that are suitable or customary for cosmetic (e.g. dermatological) and/or therapeutic applications can be used as antioxidants or preservatives.

For the purposes of the invention, antioxidants are any substances that lower the quantity of free radicals in cells and tissues. Advantageously, antioxidants are selected from the group comprising amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. alpha-carotene, beta-carotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, gamma-linoleyl, cholesteryl, glyceryl and oligoglyceryl esters) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa- and heptathionine sulfoximine) in very low acceptable doses (e.g. pmol to μmol/kg), and also (metal) chelators (e.g. alpha-hydroxy fatty acids, palmitic acid, phytic acid, lactoferrin, alpha-hydroxy acids (e.g. citric acid, lactic acid, malic acid), humic acid, gallic acid, bile extracts, tannins, bilirubin, biliverdin, EDTA, EGTA and derivatives thereof), unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate, ascorbyl glucoside), tocopherols and derivatives (e.g. vitamin E acetate), vitamin A and derivatives (vitamin A palmitate) and coniferyl benzoate from benzoin, rutic acid and derivatives thereof, flavonoids and glycosylated precursors thereof, especially quercetin and derivatives thereof, e.g. alpha-glucosylrutin, rosmaric acid, carnosol, carnosolic acid, resveratrol, caffeic acid and derivatives thereof, sinapic acid and derivatives thereof, ferulic acid and derivatives thereof, curcuminoids, chlorogenic acid and derivatives thereof, retinoids, ursolic acid, levulic acid, butyl-hydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenium methionine), superoxide dismutase, stilbenes and derivatives thereof (e.g. stilbene oxide, trans-stilbene oxide) and the derivatives of said active substances which are suitable according to the invention (salts, esters, ethers, sugars, nucleotides, nucleosides, peptides and lipids), or plant extracts or fractions with an antioxidative effect, e.g. green tea, rooibos, honeybush, grape, rosemary, sage, melissa, thyme, lavender, olive, oats, cacao, ginkgo, ginseng, licorice, honeysuckle, Sophora, Pueraria, Pinus, Citrus, Phyllanthus emblica or St John's wort, grape seeds, wheat germ and Phyllanthus emblica.

Other suitable antioxidants are coenzymes, e.g. coenzyme Q10, plastoquinone, menaquinone, ubiquinols 1-10, ubiquinones 1-10 or derivatives of these substances.

The amount of antioxidants (one or more compounds) in the formulations according to the invention is preferably 0.01 to 20 wt. %, particularly preferably 0.05 to 10 wt. % and very particularly preferably 0.2 to 5 wt. %, based on the total weight of the formulation.

If the antioxidant(s) consists (consist) of vitamin E and/or derivatives thereof, it is advantageous to choose their respective concentrations from the range 0.001 to 10 wt. %, based on the total weight of the formulation.

If the antioxidant(s) consists (consist) of vitamin A or vitamin A derivatives, or of carotenes or derivatives thereof, it is advantageous to choose their respective concentrations from the range 0.001 to 10 wt. %, based on the total weight of the formulation.

Formulations according to the invention can also contain preservatives. Preservatives which can be used are any antioxidants that are suitable or customary for cosmetic (e.g. dermatological) and/or therapeutic applications, classic preservatives (e.g. formaldehyde, glutaric dialdehyde, parabens (e.g. methyl-, ethyl-, propyl- and butylparaben), dibromodicyanobutane, imidazolidinylureas (“Germall”), isothiazolinones (“Kathon”), methylchloro-thiazolidine, methylthiazolidine, organic acids (e.g. benzoic acid, sorbic acid, salicylic acid) and salts and esters thereof, propionic acid and formic acid and salts thereof, glycols, e.g. propylene glycol, and 1,2-dihydroxyalkanes) and plant-based preservation aids, e.g. lantadin A, caryophyllene, hesperidin, diosmin, phellandrene, pigenin, quercetin, hypericin, aucubin, diosgenin, plumbagin, corlilagin, etc.

It can also be advantageous to use anti-irritants in formulations according to the invention, possible anti-irritants being any anti-inflammatory or redness-alleviating and itch-alleviating substances that are suitable or customary for cosmetic (e.g. dermatological) and/or therapeutic applications. Preferred substances are all those which reduce the amount of cytokines, interleukins, prostaglandins and/or leukotrienes in cells and tissues.

The anti-inflammatory or redness-alleviating and itch-alleviating substances used are advantageously steroidal anti-inflammatory substances of the corticosteroid type, e.g. hydrocortisone, dexamethasone, dexamethasone phosphate, methylprednisolone or cortisone, it being possible to extend the list with other steroidal anti-inflammatories. It is also possible to use non-steroidal anti-inflammatories. Examples which should be mentioned here are oxicams such as piroxicam or tenoxicam; salicylates such as aspirin, disalcid, solprin or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen or benoxaprofen, or pyrazoles such as phenylbutazone, oxyphenylbutazone, febrazone or azapropazone. A possible alternative is to use natural anti-inflammatory or redness-alleviating and itch-alleviating substances. Plant extracts, special high-activity plant extract fractions and high-purity active substances isolated from plant extracts can be used. Particular preference is afforded to extracts, fractions and active substances from camomile, Aloe vera, Commiphora species, Rubia species, Echinacea species, willow, willow-herb, oats, black and green tea, gingko, coffee, pepper, currants, tomato, vanilla, almonds, and pure substances such as, inter alia, bisabolol, apigenin-7-glucoside, boswellic acid, phytosterols, glycyrrhizinic acid, glabridin or licochalcone A.

The amount of anti-irritants (one or more compounds) in formulations according to the invention is preferably 0.01 to 20 wt. %, particularly preferably 0.03 to 10 wt. % and very particularly preferably 0.05 to 5 wt. %, based on the total weight of the formulation.

The formulations according to the invention (especially topical cosmetic formulations) can also contain moisture retention regulators and osmolytes. The following substances are examples of moisture retention regulators (moisturizers) used: sodium lactate, urea, alcohols (especially 1,2-pentanediol, 1,2-hexanediol, 1,2-octanediol, 1,2-decanediol and mixtures thereof), sorbitol, glycerol, propylene glycol, collagen, elastin or hyaluronic acid, diacyl adipates, petrolatum, ectoin, urocanic acid, lecithin, pantheol, phytantriol, lycopene, algal extract, ceramides, cholesterol, glycolipids, chitosan, chondroitin sulfate, polyamino acids and polyamino sugars, lanolin, lanolin esters, amino acids, alpha-hydroxy acids (e.g. citric acid, lactic acid, malic acid) and derivatives thereof, sugars (e.g. inositol), alpha-hydroxy fatty acids, phytosterols, triterpene acids such as betulinic acid or ursolic acid, and algal extracts. Examples of osmolytes which can be used are sugar alcohols (myoinositol, mannitol, sorbitol), quaternary amines such as taurine, choline, betaine, betaine glycine, ectoin, diglycerol phosphate, phosphorylcholine, glycerophosphorylcholines, amino acids such as glutamine, glycine, alanine, glutamate, aspartate or proline, phosphatidylcholine, phosphatidylinositol, inorganic phosphates, and polymers of said compounds, such as proteins, peptides, polyamino acids and polyols.

The formulations according to the invention (e.g. topical cosmetic formulations) also advantageously contain antimicrobial substances. The following may be mentioned as examples:

Fatty alcohols, aldehydes and acids having chain lengths of C₂ to C₄₀ which are aryl- or aryloxy-substituted, unbranched or monoalkyl- and polyalkyl-branched and saturated or monounsaturated to pentaunsaturated (up to five double or triple bonds, including mixed ene/yne compounds).

Alkanediols, dialdehydes and dicarboxylic acids having chain lengths of C₂ to C₄₀, particularly preferably of C₄ to C₁₂, which are aryl- or aryloxy-substituted, unbranched or monoalkyl- and polyalkyl-branched and saturated or monounsaturated to pentaunsaturated (up to five double or triple bonds, including mixed ene/yne compounds).

Mono- and oligoglycerides (up to 4 glycerol units) of fatty alcohols (mono- and oligoglycerol monoalkyl ethers), fatty acids (mono- and oligoglycerol monoalkyl esters), alkanediols (mono- and oligoglycerol monoalkyl ethers; bis(mono-/oligoglyceryl)alkyl diethers) and dicarboxylic acids (mono- and oligoglycerol monoalkyl esters; bis(mono-/oligoglyceryl)alkyl diesters) having chain lengths of C₂ to C₄₀ which are aryl- or aryloxy-substituted, unbranched or monoalkyl- and polyalkyl-branched and saturated or monounsaturated to pentaunsaturated (up to five double or triple bonds, including mixed ene/yne compounds). Fatty acid esters of carboxylic acids having chain lengths of C₂ to C₄₀ which are unbranched or monoalkyl- and polyalkyl-branched, saturated or monounsaturated to pentaunsaturated (up to five double or triple bonds, including mixed ene/yne compounds) and optionally also aryl- or aryloxy-substituted, with monohydric to hexahydric fatty alcohols having chain lengths of C₂ to C₄₀ which are unbranched or monoalkyl- and polyalkyl-branched, saturated or monounsaturated to pentaunsaturated (up to five double or triple bonds, including mixed ene/yne compounds) and optionally also aryl- or aryloxy-substituted.

Vegetable and animal fatty acid cuts containing fatty alcohols, aldehydes and acids having chain lengths of C₂ to C₄₀ which are unbranched or monoalkyl- and polyalkyl-branched and saturated or monounsaturated to pentaunsaturated (up to five double or triple bonds, including mixed ene/yne compounds) (e.g. coconut fatty acids, palm kernel fatty acids, wool wax acids).

Mono- and oligoglycerides of lanolin, lanolin alcohols and lanolin acids (e.g. glyceryl lanolate, neocerite), glycyrrhetinic acid and derivatives (e.g. glycyrrhetinyl stearates), natural and synthetic cardenolides (e.g. digitoxin, digoxin, digoxygenin, gitoxygenin, strophanthin and strophanthidin), natural and synthetic bufadienolides (e.g. scillaren A, scillarenin and bufotalin), sapogenins and steroid sapogenins (e.g. amyrins, oleanolic acid, digitonin, gitogenin, tigogenin and diosgenin), and steroid alkaloids of vegetable and animal origin (e.g. tomatidine, solanine, solanidine, conessine, batrachotoxin and homobatrachotoxin).

Monohalogenated and polyhalogenated nitriles, dinitriles, trinitriles or tetranitriles. Mono- and oligohydroxy fatty acids having chain lengths of C₂ to C₂₄ (e.g. lactic acid, 2-hydroxypalmitic acid), oligomers and/or polymers thereof and vegetable and animal raw materials containing them.

Acyclic terpenes: terpene hydrocarbons (e.g. ocimene, myrcene), terpene alcohols (e.g. geraniol, linalool, citronellol), terpene aldehydes and ketones (e.g. citral, pseudoionone, beta-ionone); monocyclic terpenes: terpene hydrocarbons (e.g. terpinene, terpinolene, limonene), terpene alcohols (e.g. terpineol, thymol, menthol), terpene ketones (e.g. pulegone, carvone); bicyclic terpenes: terpene hydrocarbons (e.g. carane, pinane, bornane), terpene alcohols (e.g. borneol, isoborneol), terpene ketones (e.g. camphor); sesquiterpenes: acyclic sesquiterpenes (e.g. farnesol, nerolidol), monocyclic sesquiterpenes (e.g. bisabolol), bicyclic sesquiterpenes (e.g. cardinene, selinene, vetivazulene, guaiazulene), tricyclic sesquiterpenes (e.g. santalene), diterpenes (e.g. phytol), tricyclic diterpenes (e.g. abietic acid), triterpenes (squalenoids, e.g. squalene), tetraterpenes.

Ethoxylated, propoxylated or mixed ethoxylated/propoxylated cosmetic fatty alcohols, fatty acids and fatty acid esters having chain lengths of C₂ to C₄₀ and 1 to 150 EO and/or PO units.

Antimicrobial peptides and proteins having an amino acid number of 4 to 200, e.g. skin antimicrobial peptides (SAPs), lingual antimicrobial peptides (LAPs), human beta-defensins (especially h-BD1 and h-BD2), lactoferrins and hydrolysates thereof, as well as peptides obtained therefrom, bactericidal/permeability-increasing proteins (BPIs), cationic microbial proteins (CAPs), lysozyme.

Suitable carbohydrates or “carbohydrate derivatives”, which will also be abbreviated to “carbohydrates”, are sugars and substituted sugars or compounds containing sugar residues. The sugars include especially the deoxy and dideoxy forms, N-acetylgalactosamine-, N-acetylglucosamine- and sialic acid-substituted derivatives, and sugar esters and ethers. The following are preferred:
a) monosaccharides, particularly including pentoses and hexoses,
b) disaccharides, particularly including sucrose, maltose and lactobiose,
c) oligosaccharides, particularly including trisaccharides and tetrasaccharides, and
d) polysaccharides, particularly including starch, glycogen, cellulose, dextran, tunicin, inulin, chitin, especially chitosans, chitin hydrolysates, alginic acid and alginates, plant gums, mucus, pectins, mannans, galactans, xylans, araban, polyoses, chondroitin sulfates, heparin, hyaluronic acid and glycosaminoglycans, hemicelluloses, substituted cellulose and substituted starch, especially the respective hydroxyalkyl-substituted polysaccharides.

Amylose, amylopectin, xanthan and alpha-, beta- and gamma-dextrin are particularly suitable. The polysaccharides can consist of e.g. 4 to 1,000,000 and especially 10 to 100,000 monosaccharides. The chain lengths chosen in each case are preferably such as to ensure that the active substance is soluble in or can be incorporated into the formulation in question.

Sphingolipids such as sphingosine; N-monoalkylated sphingosines; N,N-dialkylated sphingosines; sphingosine-1-phosphate; sphingosine-1-sulfate; psychosine (sphingosine beta-D-galactopyranoside); sphingosylphosphorylcholine; lysosulfatides (sphingosyl galactosylsulfate; lyso-cerebroside sulfate); lecithin; sphingomyelin; sphinganin.

It is also possible to use so-called “natural” antibacterial substances, most of which are ethereal oils. Examples of typical oils with an antibacterial action are oils from anise, lemon, orange, rosemary, wintergreen, clove, thyme, lavender, hops, citronella, wheat, lemongrass, cedarwood, cinnamon, geranium, sandalwood, violet, eucalyptus, peppermint, gum benzoin, basil, fennel, menthol and Ocmea origanum, Hydastis carradensis, Berberidaceae daceae, Ratanhiae or Curcuma longa.

Important substances with an antimicrobial action which can be found in ethereal oils are e.g. anethole, catechol, camphene, carvacrol, eugenol, eucalyptol, ferulic acid, farnesol, hinokitiol, tropolone, limonene, menthol, methyl salicylate, thymol, terpineol, verbenone, berberine, curcumin, caryophyllene oxide, nerolodol and geraniol.

It is also possible to use mixtures of said active systems or active substances, as well as combinations containing these active substances.

The amount of antimicrobial substances in the formulations is preferably 0.01 to 20 wt. % and particularly preferably 0.05 to 10 wt. %, based on the total weight of the formulations.

The formulations according to the invention (especially cosmetic formulations, including dermatological formulations) can contain deodorants, i.e. substances with a deodorizing and antiperspirant action. These include e.g. odour masking agents such as the common perfume constituents, antiperspirants based on aluminium, zirconium or zinc salts, odour absorbers, e.g. the sheet silicates described in German Offenlegungsschrift DE-P 40 09 347, including particularly montmorillonite, kaolinite, nontronite, saponite, hectorite, bentonite and smectite, and also e.g. zinc salts of ricinoleic acid. They also include bactericidal or bacteriostatic deodorizing substances, e.g. hexachlorophene, 2,4,4′-trichloro-2′-hydroxydiphenyl ether (Irgasan), 1,6-di(4-chlorophenylbiguanido)hexane (chlorhexidine), 3,4,4′-trichlorocarbanilide, and the active agents described in German Offenlegungsschriften DE-37 40 186, DE-39 38 140, DE-42 04 321, DE-42 29 707, DE-42 29 737, DE-42 37 081, DE-43 09 372 and DE-43 24 219, and contain cationic substances such as quaternary ammonium salts and odour absorbers, e.g. Grillocin® (combination of zinc ricinoleate and various additives) or triethyl citrate, optionally in combination with ion exchange resins.

The amount of deodorizing and/or antiperspirant substances in the formulations is preferably 0.01 to 20 wt. % and particularly preferably 0.05 to 10 wt. %, based on the total weight of the formulations.

The formulations according to the invention (especially cosmetic formulations) can also contain anionic, cationic, non-ionic and/or amphoteric surfactants, especially if crystalline or microcrystalline solids, e.g. inorganic micropigments, are to be incorporated into the formulations.

Anionic surfactants normally contain carboxylate, sulfate or sulfonate groups as functional groups. In aqueous solution they form negatively charged organic ions in an acidic or neutral medium. Cationic surfactants are characterized virtually exclusively by the presence of a quaternary ammonium group. In aqueous solution they form positively charged organic ions in an acidic or neutral medium. Amphoteric surfactants contain both anionic and cationic groups and accordingly behave like anionic or cationic surfactants in aqueous solution, depending on the pH. They have a positive charge in a strongly acidic medium and a negative charge in an alkaline medium. In the neutral pH range, on the other hand, they are zwitterionic. Polyether chains are typical of non-ionic surfactants. Non-ionic surfactants do not form ions in an aqueous medium.

### A. Anionic Surfactants

Anionic surfactants that can advantageously be used are acylamino acids (and salts thereof) such as
acylglutamates, e.g. sodium acylglutamate, di-TEA palmitoylaspartate and sodium caprylic/capric glutamate,
acylpeptides, e.g. palmitoyl-hydrolysed lactoprotein, sodium cocoyl-hydrolysed soya protein and sodium/potassium cocoyl-hydrolysed collagen,
sarcosinates, e.g. myristoyl sarcosine, TEA lauroylsarcosinate, sodium lauroylsarcosinate and sodium cocoylsarcosinate,
taurates, e.g. sodium lauroyltaurate and sodium methylcocoyltaurate,
acyllactylates, lauroyllactylate and caproyllactylate,
alaninates;
   carboxylic acids and derivatives, such as
lauric acid, aluminium stearate, magnesium alkanolate and zinc undecylenate,
ester-carboxylic acids, e.g. calcium stearoyllactylate, laureth-6 citrate and sodium PEG-4 lauramidocarboxylate,
ether-carboxylic acids, e.g. sodium laureth-13 carboxylate and sodium PEG-6 cocamidocarboxylate;
   phosphoric acid esters and salts, such as DEA oleth-10 phosphate and dilaureth-4 phosphate;
   sulfonic acids and salts, such as
acylisethionates, e.g. sodium/ammonium cocoylisethionate,
alkylarylsulfonates,
alkylsulfonates, e.g. sodium coco monoglyceridesulfate, sodium C₁₂₋₁₄-olefinsulfonate, sodium laurylsulfoacetate and magnesium PEG-3 cocamidosulfate,
sulfosuccinates, e.g. sodium dioctylsulfosuccinate, disodium laureth sulfosuccinate, disodium laurylsulfosuccinate and disodium MEA undecylenamidosulfosuccinate;
   and
   sulfuric acid esters such as
alkyl ether sulfate, e.g. sodium, ammonium, magnesium, MIPA and TIPA laureth sulfate, sodium myreth sulfate and sodium C12-13 pareth sulfate,
alkylsulfates, e.g. sodium, ammonium and TEA laurylsulfate.

### B. Cationic Surfactants

Cationic surfactants that can advantageously be used are
alkylamines,
alkylimidazoles,
ethoxylated amines and
quaternary surfactants:

RNH₂CH₂CH₂COO⁻ (at pH 7)

RNHCH₂CH₂COO⁻B⁺ (at pH 12), B+=arbitrary cation, e.g. Na⁺
esterquats

Quaternary surfactants contain at least one N atom that is covalently bonded to 4 alkyl or aryl groups. This produces a positive charge, irrespective of the pH. Alkylbetaine, alkylamidopropylbetaine and alkylamidopropylhydroxysulfaine are advantageous. The cationic surfactants used can also preferably be selected from the group comprising quaternary ammonium compounds, in particular benzyltrialkylammonium chlorides or bromides, e.g. benzyldimethylstearylammonium chloride, and also alkyltrialkylammonium salts, e.g. cetyltrimethylammonium chloride or bromide, alkyldimethyl-hydroxyethylammonium chlorides or bromides, dialkyldimethylammonium chlorides or bromides, alkylamidoethyltrimethylammonium ether sulfates, alkylpyridinium salts, e.g. lauryl- or cetylpyrimidinium chloride, imidazoline derivatives and compounds of a cationic nature, such as amine oxides, e.g. alkyldimethylamine oxides or alkylaminoethyldimethylamine oxides. Cetyltrimethylammonium salts can be used particularly advantageously.

### C. Amphoteric Surfactants

Amphoteric surfactants that can advantageously be used are
acyl-/dialkylethylenediamine, e.g. sodium acylamphoacetate, disodium acylamphodipropionate, disodium alkylamphodiacetate, sodium acylamphohydroxypropylsulfonate, disodium acylamphodiacetate and sodium acylamphopropionate,
N-alkylamino acids, e.g. aminopropylalkylglutamide, alkylaminopropionic acid, sodium alkylimidodipropionate and lauroamphocarboxyglycinate.

### D. Non-Ionic Surfactants

Non-ionic surfactants that can advantageously be used are
alcohols,
alkanolamides such as cocamides MEA/DEA/MIPA,
amine oxides such as cocamidopropylamine oxide,
esters formed by the esterification of carboxylic acids with ethylene oxide, glycerol, sorbitan or other alcohols,
ethers, e.g. ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers, and alkyl polyglycosides such as lauryl glucoside, decyl glycoside and coco glycoside,
sucrose esters and ethers,
polyglycerol esters, diglycerol esters and monoglycerol esters,
methyl glucose esters and esters of hydroxy acids.

The use of a combination of anionic and/or amphoteric surfactants with one or more non-ionic surfactants is also advantageous.

The surface-active substance (surfactant) or the combination of surface-active substances can be present in a concentration of between 1 and 98 wt. % in the formulations according to the invention, based on the total weight of the formulations.

Cosmetic (e.g. dermatological) or therapeutic formulations according to the invention, containing the compounds of formula (I) according to the invention or to be used according to the invention, can also take the form of emulsions.

The oily phase (lipid phase) in the formulations according to the invention (especially topical cosmetic formulations) can advantageously be selected from the following group of substances:
mineral oils (advantageously paraffin oil) and mineral waxes;
fatty oils, fats, waxes and other natural and synthetic fatty substances, preferably esters of fatty acids with alcohols of low C number, e.g. with isopropanol, propylene glycol or glycerol, or esters of fatty alcohols with alkanoic acids of low C number or with fatty acids;
alkyl benzoates (e.g. mixtures of n-dodecyl, n-tridecyl, n-tetradecyl or n-pentadecyl benzoate);
cyclic or linear silicone oils such as dimethylpolysiloxanes, diethylpolysiloxanes, diphenylpolysiloxanes and mixed forms thereof.

It is advantageous to use (natural or synthetic) esters, especially (a) esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, and (b) esters of aromatic carboxylic acids and saturated and/or unsaturated, branched and/or unbranched alcohols having a chain length of 3 to 30 C atoms. Preferred ester oils are isopropyl myristate, isopropyl palmitate, isopropyl stearate, isopropyl oleate, n-butyl stearate, n-hexyl laurate, n-decyl laurate, n-decyl oleate, isooctyl stearate, isononyl stearate, isononyl isononanoate, 3,5,5-trimethylhexyl 3,5,5-trimethylhexanoate, 2-ethylhexyl isononanoate, 2-ethylhexyl 3,5,5-trimethylhexanoate, 2-ethylhexyl 2-ethylhexanoate, cetearyl 2-ethylhexanoate, 2-ethylhexyl palmitate, 2-ethylhexyl laurate, 2-hexyldecyl stearate, 2-octyldecyl palmitate, 2-octyldodecyl palmitate, oleyl oleate, oleyl erucate, erucyl oleate, erucyl erucate, di-2-ethylhexyl 2,6-naphthalenedioate and synthetic, semisynthetic and natural mixtures of such esters, e.g. jojoba oil.

Furthermore, the oily phase can advantageously be selected from the group comprising branched and unbranched hydrocarbons and waxes, silicone oils, dialkyl ethers, the group comprising saturated or unsaturated, branched or unbranched alcohols, and also fatty acid triglycerides, specifically the triglycerol esters of saturated and/or unsaturated, branched and/or unbranched alkane-carboxylic acids having a chain length of 8 to 24 and especially 12 to 18 C atoms. The fatty acid triglycerides can advantageously be selected from the group comprising synthetic, semisynthetic and natural oils, e.g. triglycerides of capric or caprylic acid, apricot kernel oil, avocado oil, cottonseed oil, borage seed oil, thistle oil, groundnut oil, gamma-oryzanol, rose-hip oil, hemp oil, hazelnut oil, currant seed oil, coconut oil, cherry kernel oil, salmon oil, linseed oil, maize oil, macadamia nut oil, almond oil, evening primrose oil, mink oil, olive oil, palm oil, palm kernel oil, pecan nut oil, peach kernel oil, pistachio nut oil, rapeseed oil, rice germ oil, castor oil, safflower oil, sesame oil, soya oil, sunflower oil, tea tree oil, grapeseed oil or wheatgerm oil and the like. Arbitrary mixtures of such oil and wax components can also advantageously be used. In some cases it is also advantageous to use waxes, e.g. cetyl palmitate, as the sole lipid component of the oily phase; advantageously, the oily phase is selected from the group comprising 2-ethylhexyl isostearate, octyldodecanol, isotridecyl isononanoate, isoeicosane, 2-ethylhexyl cocoate, C₁₂₋₁₅-alkyl benzoate, caprylic/capric tri-glyceride and dicaprylyl ether. Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl iso-nonanoate are particularly advantageous. The hydrocarbons paraffin oil, squalane and squalene can also advantageously be used. Advantageously, the oily phase can further contain cyclic or linear silicone oils or consist entirely of such oils, although it is preferable to use other oily phase components in addition to the silicone oil(s). Cyclomethicone (e.g. decamethylcyclopentasiloxane) can advantageously be used as a silicone oil. However, other silicone oils can also advantageously be used, examples being undecamethylcyclotrisiloxane, poly-dimethylsiloxane and poly(methylphenylsiloxane). Furthermore, mixtures of cyclomethicone and isotridecyl isononanoate and of cyclomethicone and 2-ethylhexyl isostearate are particularly advantageous.

The aqueous phase of formulations according to the invention (especially topical cosmetic formulations) that take the form of an emulsion can advantageously comprise alcohols, diols or polyols of low C number, as well as ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low C number, e.g. ethanol, isopropanol, 1,2-propanediol and glycerol, and in particular one or more thickeners, which can advantageously be selected from the group comprising silicon dioxide, aluminium silicates such as bentonites, polysaccharides or derivatives thereof, e.g. hyaluronic acid, guar kernel flour, xanthan gum, hydroxy-propyl methyl cellulose or allulose derivatives, and particularly advantageously from the group comprising polyacrylates, preferably a polyacrylate from the group comprising the so-called carbopols, e.g. carbopols of types 980, 981, 1382, 2984 and 5984, in each case on their own or in combination, or from the group comprising polyurethanes, and also alpha- or beta-hydroxy acids, preferably lactic acid, citric acid or salicylic acid, as well as emulsifiers, which can advantageously be selected from the group comprising ionic, non-ionic, polymeric, phosphate-containing and zwitterionic emulsifiers.

Formulations according to the invention that take the form of an emulsion advantageously comprise one or more emulsifiers. O/W emulsifiers can, for example, advantageously be selected from the group comprising polyethoxylated or polypropoxylated or polyethoxylated and polypropoxylated products, e.g.:
fatty alcohol ethoxylates,
ethoxylated wool wax alcohols,
polyethylene glycol ethers of the general formula

R—O—(—CH₂—CH₂—O—)ₙ—R′,
fatty acid ethoxylates of the general formula

R—COO—(—CH₂—CH₂—O—)ₙ—H,
etherified fatty acid ethoxylates of the general formula

R—COO—(—CH₂—CH₂—O—)ₙ—R′,
esterified fatty acid ethoxylates of the general formula

R—COO—(—CH₂—CH₂—O—)ₙ—C(O)—R′,
polyethylene glycol glycerol fatty acid esters,
ethoxylated sorbitan esters,
cholesterol ethoxylates,
ethoxylated triglycerides,
alkyl ether carboxylic acids of the general formula

R—COO—(—CH₂—CH₂—O—)ₙ—OOH, where n is a number from 5 to 30,
polyoxyethylene sorbitol fatty acid esters,
alkyl ether sulfates of the general formula

R—O—(—CH₂—CH₂—O—)ₙ—SO₃—H,
fatty alcohol propoxylates of the general formula

R—O—(—CH₂—CH(CH₃)—O—)ₙ—H,
polypropylene glycol ethers of the general formula

R—O—(—CH₂—CH(CH₃)—O—)ₙ—R′,
propoxylated wool wax alcohols,
etherified fatty acid propoxylates

R—COO—(—CH₂—CH(CH₃)—O—)ₙ—R′,
esterified fatty acid propoxylates of the general formula R—COO—(—CH₂—CH(CH₃)—O—)ₙ—C(O)—R′,
fatty acid propoxylates of the general formula

R—COO—(—CH₂—CH(CH₃)—O—)ₙ—H,
polypropylene glycol glycerol fatty acid esters,
propoxylated sorbitan esters,
cholesterol propoxylates,
propoxylated triglycerides,
alkyl ether carboxylic acids of the general formula

R—O—(—CH₂—CH(CH₃)—O—)ₙ—CH₂—COOH,
alkyl ether sulfates, or the acids on which these sulfates are based, of the general formula

R—O—(—CH₂—CH(CH₃)—O—)ₙ—SO₃—H,
fatty alcohol ethoxylates/propoxylates of the general formula R—O—Xₙ—Yₘ—H,
polypropylene glycol ethers of the general formula

R—O—Xₙ—Yₘ—R′,
etherified fatty acid propoxylates of the general formula R—COO—Xₙ—Yₙ—R′,
fatty acid ethoxylates/propoxylates of the general formula R—COO—Xₙ—Yₘ—H.

According to the invention, the polyethoxylated or polypropoxylated or polyethoxylated and polypropoxylated O/W emulsifiers used are particularly advantageously selected from the group comprising substances having HLB values of 11 to 18 and very particularly advantageously having HLB values of 14.5 to 15.5, if the O/W emulsifiers contain saturated radicals R and R′. If the O/W emulsifiers contain unsaturated radicals R and/or R′, or if isoalkyl derivatives are present, the preferred HLB value of such emulsifiers can also be lower or higher.

It is advantageous to select the fatty alcohol ethoxylates from the group comprising ethoxylated stearyl alcohols, cetyl alcohols and cetylstearyl alcohols (cetearyl alcohols). The following are particularly preferred:

polyethylene glycol (n) stearyl ethers (steareth-n) where
n=13-20,
polyethylene glycol (n) cetyl ethers (ceteth-n) where n=13-20,
polyethylene glycol (n) isocetyl ethers (isoceteth-n) where n=13-20,
polyethylene glycol (n) cetylstearyl ethers (ceteareth-n)
where n=13-20,
polyethylene glycol (m) isostearyl ethers (isosteareth-m) where m=12-20,
polyethylene glycol (k) oleyl ethers (oleth-k) where k=12-15,
polyethylene glycol (12) lauryl ether (laureth-12),
polyethylene glycol (12) isolauryl ether (isolaureth-12).

It is also advantageous to select the fatty acid ethoxylates from the following group:

polyethylene glycol (n) stearates where n=20-25,
polyethylene glycol (m) isostearates where m=12-25,
polyethylene glycol (k) oleates where k=12-20.

Sodium laureth-11 carboxylate can advantageously be used as an ethoxylated alkyl ether carboxylic acid or a salt thereof. Sodium laureth-1-4 sulfate can advantageously be used as an alkyl ether sulfate. Polyethylene glycol (30) cholesteryl ether can advantageously be used as an ethoxylated cholesterol derivative. Polyethylene glycol (25) soyasterol has also proved useful.

Polyethylene glycol (60) evening primrose glycerides can advantageously be used as ethoxylated triglycerides.

It is also advantageous to select the polyethylene glycol glycerol fatty acid esters from the group comprising polyethylene glycol (n) glyceryl laurates where n=20-23, polyethylene glycol (6) glyceryl caprylate/caprate, polyethylene glycol (20) glyceryl oleate, polyethylene glycol (20) glyceryl isostearate and polyethylene glycol (18) glyceryl oleate/cocoate.

It is likewise favourable to select the sorbitan esters from the group comprising polyethylene glycol (20) sorbitan monolaurate, polyethylene glycol (20) sorbitan monostearate, polyethylene glycol (20) sorbitan monoisostearate, polyethylene glycol (20) sorbitan monopalmitate and polyethylene glycol (20) sorbitan monooleate.

The following can be used as advantageous W/O emulsifiers: fatty alcohols having 8 to 30 carbon atoms, monoglycerol esters of saturated and/or unsaturated, branched and/or unbranched alkanecarboxylic acids having a chain length of 8 to 24 C atoms, especially 12 to 18 C atoms, diglycerol esters of saturated and/or unsaturated, branched and/or unbranched alkanecarboxylic acids having a chain length of 8 to 24 C atoms, especially 12 to 18 C atoms, monoglycerol ethers of saturated and/or unsaturated, branched and/or unbranched alcohols having a chain length of 8 to 24 C atoms, especially 12 to 18 C atoms, diglycerol ethers of saturated and/or unsaturated, branched and/or unbranched alcohols having a chain length of 8 to 24 C atoms, especially 12 to 18 C atoms, propylene glycol esters of saturated and/or unsaturated, branched and/or unbranched alkanecarboxylic acids having a chain length of 8 to 24 C atoms, especially 12 to 18 C atoms, and sorbitan esters of saturated and/or unsaturated, branched and/or unbranched alkanecarboxylic acids having a chain length of 8 to 24 C atoms, especially 12 to 18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprate and glyceryl monocaprylate.

It is also possible to use mixtures of said active systems.

The total amount of compounds of formulae (III), (IV) and (V), UV absorbers and (other) skin-lightening substances in the formulations according to the invention is preferably 0.01 to 20 wt. % and particularly preferably 0.05 to 15 wt. %, based on the total weight of the formulation.

A drug and/or formulation according to the invention is preferably prepared by extracting wood, particularly preferably comminuted wood, for up to 72 h with an extractant which comprises water, ethyl acetate, an alcohol or a ketone selected from the group comprising methanol, ethanol, n-propanol, isopropanol and/or acetone, and mixtures of two or more of these substances. Particularly preferred extractants contain methanol, ethanol, acetone or mixtures of two or more of these substances.

Maackiain is found inter alia in the wood of Maackia species, e.g. M. amurensis and M. tenuifolia. However, maackiain and other compounds of formulae (III) and/or (IV) are also widespread in other plants and other plant parts, e.g. in Andira inermis, Artemisia indica, Baphia nitida, Baptisia tinctoria, Cicer species, e.g. C. arietinum, C. bijugum, C. echinospermum and C. reticulatum, Dalbergia species, e.g. D. oliveri, D. spruceana and D. sericea, Derris elliptica, Machaerium aristulatum, Ononis vaginalis, Osteophloeum platyspermum, Petalostemon purpureus, Pisum sativum, Pterocarpus species, Sophora species, e.g. S. japonica, S. subprostata, S. angustifolia and S. flavescens, Spatholobus suberectus, Swartzia madagascariensis, Tephrosia purpurea, Trifolium species such as T. pratense, T. hybridum and T. repens, Ulex species, e.g. U. minor and U. jussiaei, Virgilia oroboides and other plants.

Rosewood (Dalbergia variabilis) extracts contain medicarpin and variabilin and likewise showed a very good activity. However, it is also possible to use other woods or plants containing compounds of formulae (III) and/or (IV):

Andira inermis, Albizzia procera, Alysicarpus sp., Amorpha californica, Apios tuberosa, Artemisia Indica, Astragalus species, e.g. A. membranaceus and A. mongholicus, Baphia nitida, Bituminaria species, e.g. B. bituminosa and B. morisiana, Bolusanthus speciosus, Bowdichia nitida, Brya ebenus, Calopogonium mucunoides, Cladrastis platycarpa, Crotalaria species, e.g. C. pallida, C. assamica, C. pallida, C. barbata and C. mucronata, Dalbergia species, e.g. D. spruceana, D. odorifera, D. stevensonii, D. oliveri, D. sericea, D. nitidula and D. decipularis, Dalea filiciformis, Derris species, e.g. D. elliptica and D. oblonga, Desmodium gangeticum, Dolichos biflorus, Erythrina species such as Erythrina crista-galli, E. variegata, E. abyssinica, E. milbraedii, E. glauca, E. orientalis, E. sandwicensis, E. suberosa and E. poeppigiana, Euchresta horsfieldii, Flemingia chappar, Glycine species, e.g. G. clandestina, G. max, G. falcata, G. latrobeana, G. soja, G. canescens, G. tabacina and G. tomentella, Glycyrrhiza species, e.g. G. aspera, G. glabra and G. uralensis, Harpalyce brasiliana, Hedysarum multijugum, Lablab niger, Lespedeza species, e.g. L. homoloba and L. cyrtobotrya, Lonchocarpus species, e.g. L. laxiflorus and L. urucu, Maackia amurensis, Machaerium species such as M. villosum and M. vestitum, Medicago sativa, Melilotus species, e.g. M. alba and M. indica, Millettia species, e.g. M. pervilleana and M. pulchra, Mundulea striata, Myroxylon peruiferum, Neorautanenia species, e.g. N. ficifolia, N. edulis and N. amboensis, Nissolia fruticosa, Oroxylum indicum, Osteophloeum platyspermum, Pachyrrhizus species, e.g. P. erosus and P. erectus, Pericopsis species, e.g. P. angolensis and P. achliebenii, Petalostemon purpureus, Phaseolus species, e.g. P. lunatus, P. vulgaris, P. aureus, P. calcaratus, Pisum sativum, Platymiscium trinitatis, Psophocarpus tetragonolobus, Pterocarpus species such as P. santalinus and P. soyauxii, Pueraria species, e.g. P. tuberosa, P. mirifica and P. phaseoloides, Sophora species, e.g. S. prostrata, S. franchetiana, S. arizonica, S. flavescens, S. japonica, S. franchetiana, S. tomentosa, S. secundiflora, S. tetraptera, S. junceum, Swartzia species such as S. madagascariensis, S. laevicarpa, S. ulei and S. leiocalycina, Tephrosia species such as T. emoroides, T. aequilata, T. hildebrandtii and T. bidwilli, Trifolium species, e.g. T. hybridum, T. cherleri, T. pallescens, T. repens, Ulex species such as U. europaeus and U. parviflorus, Vigna unguiculata and Virgilia oroboides, as well as other species of the family Leguminosae, including especially the subfamily Papilionoideae. Naturally, synthetic compounds of formulae (III) and/or (IV) can also be used for the same purpose.

To prepare a drug and/or formulation according to the invention, especially one that contains a compound of formula (V), the following woods can also be extracted in the manner described above: Acacia species, e.g. A. crombei, A. peuce, A. fasciculifera and A. carnei, Cassine species, e.g. C. papillosa and C. transvaalensis, Caesalpinia species, e.g. C. pulcherrima, Ceratostigma minus, Colophospermum mopane, Distemonanthus benthamianus, Elaeodendron balae, Entandrophragma cylindricum, Goniorrhachis marginata, Iris bungei, Trachylobium species, e.g. T. verrucosum, Woodsia scopulina and Umtiza listeriana.

It is also possible to extract mixtures of two or more woods of the species listed above. It is preferable to extract heartwood.

Particularly preferred extracts are prepared with extractants that contain ethanol as the alcohol constituent. In contrast to extractants containing methanol, for example, these extractants are comparatively safe to handle and easy to obtain in industrially adequate quality. In particularly preferred processes for the preparation of correspondingly preferred extracts, the extractant contains only one alcohol, preferably ethanol. Those skilled in the art are aware that, especially when using industrial alcohol, the extractant can also contain other constituents as impurities, such impurities being insignificant for the success of the extraction process according to the invention.

The ratio of the weight of extractant to the dry weight of wood is chosen to give the mixture a good stirrability. It is preferably adjusted so that the weight of extractant is at least 5 times, preferably not more than 50 times and particularly preferably 5 to 20 times the dry weight of wood. Very particularly preferably, the weight of extractant used for the extraction is 7 to 15 times the dry weight of wood.

The extraction time for carrying out step b) is at most 72 hours, but can also be shorter. If the extraction times are particularly short, only a very dilute extract is obtained in step b). It is therefore preferable to extract the wood in step b) for at least 1 h and especially for at least 2 h. The extraction time required to obtain an extract with a for use in the preparation of cosmetic and/or pharmaceutical formulations or drugs is preferably at most 24 h and particularly preferably at most 4 h. The required extraction time is chosen according to the quality of the plant material to be extracted and according to the other extraction conditions, especially the temperature. At high extraction temperatures, the extraction time is preferably 1 h to 6 h and particularly preferably 2 h to 4 h.

The extraction temperature is adjusted according to the extractant used and is normally at least 25° C., preferably 40-150° C. and particularly preferably 50-120° C.

The extracts according to the invention can be processed further to extracts according to the invention in solid form, the preparative process according to the invention being extended by the following steps:

b) optional addition of a pharmaceutically and/or cosmetically acceptable solid excipient to the extract, and
c) drying of the extract, to which the excipient has optionally been added in step b), until the residual extractant content is at most 5 wt. %, based on the total weight of the extract obtained in step c).

It is also possible according to the invention for step b) to be omitted, in which case the powder obtained is more highly concentrated than in the case where a pharmaceutically and/or cosmetically acceptable excipient is added. A pharmaceutically or cosmetically acceptable solid is one which is at least non-toxic to the organism on which it is to be used. Preferred cosmetically or pharmaceutically acceptable solids are pulverulent maltodextrin, lactose, silicon dioxide or glucose, and mixtures of two or more of these solids.

According to the invention, a particularly preferred preparative process is one in which the extract, optionally together with a pharmaceutically and/or cosmetically acceptable excipient, preferably such as maltodextrin and/or glucose, is processed further to a powder by spray drying; the corresponding extract is also particularly preferred. This method makes it possible to prepare extracts according to the invention with good keeping properties, which are particularly suitable for further processing for the uses according to the invention which are described here. Moreover, by adjusting the mixing ratio of the extract and the pharmaceutically and/or cosmetically acceptable excipient, the final concentration of the active substances contained in the powdered extract can be adjusted in an advantageously simple manner.

Furthermore, according to the invention, the solid or liquid wood extract can also be processed further to a liquid formulation by being mixed with a solvent selected from the group comprising glycerol, 1,2-propylene glycol, 1,3-butylene glycol, ethanol, water and mixtures of two or more of said solvents with water and vegetable oils or neutral oil. Such extracts prepared according to the invention are particularly suitable for further processing for cosmetic purposes. These formulations according to the invention can optionally be prepared with the addition of a preservative, solubilizer or antioxidant.

According to the invention, the solid or liquid extract, or the liquid or solid formulation containing extract, can also be processed further by encapsulation. According to the invention, the extract and/or the liquid or solid formulation containing it, as described above, is encapsulated with a solid coating material preferably selected from starches, degraded or chemically or physically modified starches (especially dextrins and maltodextrins), gelatins, gum arabic, agar-agar, gum ghatti, gellan gum, modified and unmodified celluloses, pullulan, curdlan, carrageenans, alginic acid, alginates, pectin, inulin, xanthan gum and mixtures of two or more of said substances. Encapsulation of the extract with liposomes can also be advantageous.

Furthermore, an active substance-enriched fraction or the pure active substance can be obtained from the extract by suitable processes, examples being successive extraction (i.e. consecutive extractions of the material with different extractants), redissolution, liquid-liquid partition, precipitation or crystallization, membrane filtration and chromatographic separation processes. The active substance fraction according to the invention and the active substance itself can likewise be processed further to an active substance according to the invention in solid or liquid form, or by encapsulation.

For use, the topical formulations according to the invention, especially formulations for lightening the skin and hair, are applied to the skin and/or hair in in a sufficient amount and in a manner customary for cosmetic preparations.

The invention is described in greater detail below with the aid of the Examples, which are not intended to limit the

extent of protection defined by the Claims. Unless indicated otherwise, all the data are by weight.

### EXAMPLE 1

### Preparation of Rosewood Ethanol Extract

335 g of ethanol are added to 68 g of comminuted rosewood and the mixture is stirred for two hours under reflux at a temperature of 70-80° C. After the extraction mixture has cooled to room temperature, it is passed through a fluted filter and the clear filtrate is concentrated to dryness under vacuum on a rotary evaporator to give 2.5 g (yield 3.6%) of rosewood extract.

In another preparation, 575 g of acetone are added to 53 g of comminuted rosewood and the mixture is stirred for two hours under reflux at a temperature of 50-60° C. After the extraction mixture has cooled to room temperature, it is passed through a fluted filter and the clear filtrate is concentrated to dryness under vacuum on a rotary evaporator to give 2.1 g (yield 4.0%) of rosewood extract (containing variabilin and medicarpin).

Characterization by HPLC fingerprint analysis: column: YMC ODS-AQ, 5 μm, 150×3 mm with precolumn, temperature: 40° C., flow rate: 0.6 ml/min, acetonitrile/water with 0.1% formic acid gradient, injection volume: 5 μl, detection wavelength: 280 nm.

### EXAMPLE 2

### Preparation Amaranth Wood 1:1 Ethanol/Water Extract

665 g of a 1:1 (w/w) ethanol/water mixture are added to 62 g of comminuted amaranth wood and the mixture is stirred for two hours under reflux at a temperature of 70-80° C. After the extraction mixture has cooled to room temperature, it is passed through a fluted filter and the clear filtrate is concentrated to dryness under vacuum on a rotary evaporator to give 4.5 g (yield 7.3%) of amaranth extract containing 22.8% of peltogynol (peltogynol and peltogynol B).

Characterization by HPLC fingerprint analysis and quantitative determination of the peltogynol content: column: YMC ODS-AQ, 5 μm, 150×3 mm with precolumn, temperature: 40° C., flow rate: 0.6 ml/min, acetonitrile/water containing 0.1% formic acid gradient, injection volume: 5 μl, detection wavelength: 280 nm.

### Cell Culture and Irradiation

HaCaT keratinocytes were cultivated in DMEM containing 10% of foetal calf serum. The cells were irradiated with UVB in PBS (phosphate buffered saline). For irradiation with UVB we used a TL20W/12RS lamp, which contains four parallel tubes (Philips, Eindhoven, The Netherlands) and emits the bulk of its energy in the UVB range (290-320 nm). The emission peak of the lamp is at 310 nm. Control cells were subjected to the same treatment but were not irradiated. To inhibit the AhR, the cells were treated with the test substances 1 h prior to irradiation.

Transfection of HaCaT Cells with Plasmid pEGFP-AhR

HaCaT cells were plated out on compartmentalized microscope slides at a cell density of 5×10⁴ cells/compartment. Some were pretreated for 1 h with the test substances. After 24 h these were transfected with plasmid pEGFP-AhR by means of the FuGene 6 transfection reagent (Roche, Mannheim, Germany) according to the manufacturer's instructions. After a further 24 h the transfected cells were irradiated with 100 J/m² of UVB. After 40 min the cells were fixed for 10 min with 4% paraformaldehyde and washed with PBS. The slides were dried and covered with Vectashield mounting medium (Vector Laboratories, Burlingame, Calif., USA). The GFP-coupled AhR was visualized by means of a fluorescence microscope (Olympus, Hamburg, Germany) and photographed with a ColorView XS digital camera (Olympus).

RNA Preparation, cDNA Synthesis and Real Time RT-PCR

HaCaT cells were irradiated with 100 J/m² of UVB. Some cells were pretreated with the test substances 1 h prior to irradiation. After 4 h the RNA was prepared with an RNeasy kit (Qiagen, Hilden, Germany) according to the manufacturer's instructions. Reverse transcription was carried out as described (Arch. Toxicol. (2005) PMID 16205913). PCR fragments were amplified by means of real-time PCR in a LightCycler (Roche, Mannheim, Germany). The PCR mix was composed of 1/10 by volume of QuantiTect® SYBR Green PCR Master Mix (Qiagen, Hilden, Germany), 0.5 μmol/l of the appropriate primer, 2 μl of cDNA and DEPC-treated (diethyl pyrocarbonate-treated) H₂O in a final volume of 20 μl. The PCR began by heating for 15 minutes at 95° C. to activate the DNA polymerase. The PCR conditions were as follows: 40 cycles of 15 sec at 94° C. for denaturing, 25 sec at 60° C. for primer attachment, 30 sec at 72° C. for extension and 2 sec at 72° C. for fluorescence measurement. PCR primers for human CYP1A1 had the following sequences: 5′-TAGACACTGATCTGGCTG-CAG for the forwards primer and 5′-GGGAAGGCTCCATCAGCATC for the reverse primer (Cancer Res. 1990, 50, 4315), which formed a 146 bp fragment after amplification. The PCR products were quantified via a fragment-specific standard curve using LightCycler software 3. Standard curves were established with 102 to 106 CYP1A1 cDNA copies/μl and amplified as described above.

### Results

Quantification of AhR Inhibition Via Determination of CYP1A1-mRNA

TABLE 1

| | | | CYP1A1 inhibition in |
|---|---|---|---|
| Substance | Concentration | | presence of UVB light* |
| Maackiain | 0.35 | mM | 60% |
| Extract of Example 1 | 0.001 | wt. % | 25% |
| (containing variabilin, | | | |
| medicarpin) | | | |
| Extract of Example 2 | 0.0001 | wt. % | 55% |
| (containing peltogynol) | | | |
| Peltogynol | 0.0001 | wt. % | 60% |

*relative to the control (PBS without test substances, containing 0.1% of DMSO, irradiated with UVB)

Formulation 1: “Water-in-Oil” Emulsion with UVA/B Broadband Protection
Formulation 2: “Oil-in-Water” Emulsion with UVA/B Broadband Protection
Formulation 3: “Oil-in-Water” Emulsion with UVA/B Broadband Protection
Formulation 4: Oil-Free Sun Spray with UVA/B Broadband Protection
Formulation 5: Balm with UVA/UVB Protection
Formulation 6: Aerosol Foam with UVB/UVA Protection
Formulation 7: non-aerosol foam
Formulation 8: Shampoo with UVB Protection of Cells
Formulation 9: Hair Conditioner with UVB/UVA Protection
Formulation 10: O/W Day Cream with UVB Protection of Cells
Formulation 11: W/O Night Cream with UVB Protection of Cells

TABLE 2
Compositions of formulations according to the invention (Examples 1-11)

| NAME OF RAW | | | | | | | | | | | | |
|---|---|---|---|---|---|---|---|---|---|---|---|---|
| MATERIAL | | Wt. % | | | | | | | | | | |
| (MANUFACTURER) | INCI | 1 | 2 | 3 | 4 | 5 | 6 | 7 | 8 | 9 | 10 | 11 |
| AhR antagonists | | | | | | | | | | | | |
| Extract of Example 1 | | | | 0.5 | 0.2 | 1.0 | 0.25 | | 0.1 | 0.2 | | 0.2 |
| (containing | | | | | | | | | | | | |
| variabilin, | | | | | | | | | | | | |
| medicarpin) | | | | | | | | | | | | |
| Extract of Example 2 | | | 3.0 | | | | 0.25 | 0.5 | | | 1.0 | |
| (containing | | | | | | | | | | | | |
| peltogynol) | | | | | | | | | | | | |
| Peltogynol | | 0.05 | | | 0.05 | | | | 0.1 | | | 0.05 |
| Maackiain | | 0.05 | | 0.05 | | | | | | | | 0.05 |
| Other ingredients | | | | | | | | | | | | |
| Abil 100 ® | Dimethicone | | 1.0 | 0.3 | | | | | | | | |
| (Goldschmidt) | | | | | | | | | | | | |
| Abil 350 | Dimethicone | | | | | | | | | | 0.5 | |
| (Degussa- | | | | | | | | | | | | |
| Goldschmidt) | | | | | | | | | | | | |
| Alpha-bisabolol, | Bisabolol | | | | | | | | | | 0.3 | 0.2 |
| natural (Symrise) | | | | | | | | | | | | |
| Aloe Vera Gel | Water (Aqua), | | | | | | | | | | | |
| Concentrate 10/1 | Aloe | | | | | | | | | | | |
| (Symrise) | Barbadensis | | | | | | | | | | | |
| | Leaf Juice | | | | | | | | | | | |
| Alugel 34 TH | Aluminium | | | | | | | | | | | 1.0 |
| (Baerlocher) | Stearate | | | | | | | | | | | |
| Arbutin (Sabinsa) | β-Arbutin | | | | | | | | | | 1.0 | |
| Arlypon F | Laureth-2 | | | | | | | | 2.0 | | | |
| Baysilone oil M10 | Dimethicone | | | | | | | 1.0 | | | | |
| (GE Bayer) | | | | | | | | | | | | |
| Baysilone oil PK 20 | Phenyl | | | | | 5.0 | | | | | | |
| (GE Bayer) | Trimethicone | | | | | | | | | | | |
| Bentone Gel M IO ® | Mineral oil and | | 3.0 | | | | | | | | | |
| (Rheox) | Quaternium-18- | | | | | | | | | | | |
| | hectorite and | | | | | | | | | | | |
| | Propylene | | | | | | | | | | | |
| | carbonate, | | | | | | | | | | | |
| | Glyceryl | | | | | | | | | | | |
| | stearate and | | | | | | | | | | | |
| | Cetyl alcohol | | | | | | | | | | | |
| Alpha-bisabolol | Bisabolol | 0.1 | | 0.1 | | 0.2 | 0.1 | 0.1 | 0.1 | | | |
| (Symrise) | | | | | | | | | | | | |
| 1,3-Butylene glycol | 1,3-Butylene | | | 3.0 | | | | | | | | |
| | glycol | | | | | | | | | | | |
| Carbopol 2050 ® | Carbomer | | | 0.2 | | | 0.1 | | | | | |
| (B. F. Goodrich) | | | | | | | | | | | | |
| Carbopol ETD 2001 | Carbomer | | | | | 0.5 | | | | | | |
| (Noveon) | | | | | | | | | | | | |
| Ceramide 2 | Ceramide 2 | | | | | | | | | | 0.1 | |
| (Sederma) | | | | | | | | | | | | |
| Ceramide SL (Sino | Hydroxyethyl | | | | | | | | | | | 0.1 |
| Lion) | Palmityl | | | | | | | | | | | |
| | Oxyhydroxypropyl | | | | | | | | | | | |
| | Palmitamide | | | | | | | | | | | |
| Cetiol SN ® (Cognis) | Cetyl and | 5.0 | 4.0 | 5.0 | | | | | | | | |
| | Stearyl | | | | | | | | | | | |
| | isononanoate | | | | | | | | | | | |
| Cetiol OE (Cognis) | Dicaprylyl Ether | | | | | | | 3.0 | | | | |
| Citric Acid | Citric Acid | | | | | | | | | 0.1 | | |
| Copherol 1250 ® | Tocopherol | 1.0 | | 0.5 | | 0.5 | 0.5 | 0.5 | | | | |
| (Cognis) | acetate | | | | | | | | | | | |
| Corapan TQ ® | Diethylhexyl | | | 3.0 | | | | | | | | |
| (Symrise) | 1,6-Naphthalate | | | | | | | | | | | |
| Crinipan ® AD | Climbazole | | | | | | | | 0.5 | | | |
| (Symrise) | | | | | | | | | | | | |
| Crotein Q (Croda) | Hydroxypropyl | | | | | | | | | 1.0 | | |
| | trimonium | | | | | | | | | | | |
| | Hydrolysed | | | | | | | | | | | |
| Cutina CBS ® | Glyceryl | | 2.0 | | | | | | | | | |
| (Cognis) | stearate and | | | | | | | | | | | |
| | Cetyl alcohol | | | | | | | | | | | |
| | and Stearyl | | | | | | | | | | | |
| | alcohol and | | | | | | | | | | | |
| | Cetyl palmitate | | | | | | | | | | | |
| | and Coconut | | | | | | | | | | | |
| | glyceride | | | | | | | | | | | |
| Dehymuls PGPH ® | Polyglycerol-2 | 3.0 | | | | | | | | | | |
| (Cognis) | Dipolyhydroxystearate | | | | | | | | | | | |
| Dehyquart SP | Quaternium-52 | | | | | | | | | 0.5 | | |
| Dehyton K | Cocamidopropyl | | | | | | | | 12.0 | | | |
| | Betaine | | | | | | | | | | | |
| Dow Corning ® 193 | Dimethicone- | | | | 1.0 | | | | | | | |
| (Dow Corning) | Polyol | | | | | | | | | | | |
| Dow Corning 200 | Dimethicone | | | | | | | | | | | |
| Fluid (Dow Corning) | | | | | | | | | | | | |
| D-Panthenol (BASF) | Panthenol | | | | 0.5 | | | 0.5 | 0.4 | | | |
| Dracorin 100 s.e. ® | Glyceryl | | 3.0 | | | | | | | | | |
| (Symrise) | stearate (and) | | | | | | | | | | | |
| | PEG-100 | | | | | | | | | | | |
| | Stearate | | | | | | | | | | | |
| Dracorin CE | Glyceryl | | | | | | | | | | 5.0 | |
| (Symrise) | Stearate Citrate | | | | | | | | | | | |
| Dragocid Liquid | Phenoxyethanol | 0.3 | 0.3 | 0.3 | | 0.3 | 0.3 | 0.3 | | 0.5 | | 0.8 |
| (Symrise) | (and) | | | | | | | | | | | |
| | Methylparaben | | | | | | | | | | | |
| | (and) | | | | | | | | | | | |
| | Ethylparaben | | | | | | | | | | | |
| | (and) | | | | | | | | | | | |
| | Butylparaben | | | | | | | | | | | |
| | (and) | | | | | | | | | | | |
| | Propylparaben | | | | | | | | | | | |
| | (and) | | | | | | | | | | | |
| | Isobutylparaben | | | | | | | | | | | |
| Drago-Beta-Glucan | Water (Aqua), | | | | | | | | | | 0.3 | |
| (Symrise) | Butylene Glycol, | | | | | | | | | | | |
| | Glycerol, Avena | | | | | | | | | | | |
| | Sativa (Oat) | | | | | | | | | | | |
| | Kernel Extract | | | | | | | | | | | |
| Dragoderm | Glycerol, | | | | | | | | 2.0 | | | |
| (Symrise) | Triticum Vulgare | | | | | | | | | | | |
| | (Wheat) Gluten, | | | | | | | | | | | |
| | Water (Aqua) | | | | | | | | | | | |
| Dragophos S | Sodium | | | | | | | | | | | |
| (Symrise) | Dihydroxycetyl | | | | | | | | | | | |
| | Phosphate | | | | | | | | | | | |
| Dragorin GMS | Glyceryl | | | 2.0 | | | 2.0 | | | | | |
| (Symrise) | Stearate | | | | | | | | | | | |
| Dragosan W/O | Polyglyceryl-3- | | | | | | | | | | | 1.0 |
| Liquid (Symrise) | Polyricinoleate, | | | | | | | | | | | |
| | Sorbitan | | | | | | | | | | | |
| | Isostearate | | | | | | | | | | | |
| Dragosan W/O P | Sorbitan | | | | | | | | | | | 6.0 |
| (Symrise) | Isostearate, | | | | | | | | | | | |
| | Hydrogenated | | | | | | | | | | | |
| | Castor Oil, | | | | | | | | | | | |
| | Ceresin, | | | | | | | | | | | |
| | Beeswax (Cera | | | | | | | | | | | |
| | Alba) | | | | | | | | | | | |
| Dragoxat EH | Ethylhexyl | | | | | | | | | | 3.0 | |
| (Symrise) | Ethylhexanoate | | | | | | | | | | | |
| Edeta BD ® (BASF) | Disodium EDTA | 0.1 | 0.1 | 0.1 | | 0.1 | 0.1 | 0.1 | | | | |
| Emulgin B2 ® | Ceteareth-20 | | 1.0 | | | | | | | 0.7 | | |
| (Cognis) | | | | | | | | | | | | |
| Emulsiphos | Cetyl | | | 1.5 | | | 1.5 | | | | | |
| (Symrise) | phosphate, | | | | | | | | | | | |
| | Hydrogenated | | | | | | | | | | | |
| | Palm glycerides | | | | | | | | | | | |
| Ethanol (96%) | Ethyl alcohol | | | | 13.0 | 5.0 | | | | | | |
| Euxyl K 100 ® | Methylchloroiso- | | | | 0.1 | | | | | | | |
| (Schülke & Mayr) | thiazolinone, | | | | | | | | | | | |
| | Methylisothiazolinone | | | | | | | | | | | |
| Extrapon Aloe Vera | Aqua, Aloe | | | | 1.0 | | | | | | | |
| (Symrise) | Barbadensis, | | | | | | | | | | | |
| | Propylene | | | | | | | | | | | |
| | Glycol, Alcohol | | | | | | | | | | | |
| Extrapon Kamille | Glycerol, Water | | | | 1.0 | | | | | | | |
| (Symrise) | (Aqua), | | | | | | | | | | | |
| | Chamomilla | | | | | | | | | | | |
| | Recutita | | | | | | | | | | | |
| | (Matricaria) | | | | | | | | | | | |
| | Flower Extract | | | | | | | | | | | |
| Extrapon Hamamelis | Propylene | | | | 1.0 | | | | | | | |
| (Symrise) | Glycol, | | | | | | | | | | | |
| | Hamamelis | | | | | | | | | | | |
| | Virginiana | | | | | | | | | | | |
| | (Witch Hazel) | | | | | | | | | | | |
| | Water, Water | | | | | | | | | | | |
| | (Aqua), Alcohol, | | | | | | | | | | | |
| | Hamamelis | | | | | | | | | | | |
| | Virginiana | | | | | | | | | | | |
| | (Witch Hazel) | | | | | | | | | | | |
| | Bark/Leaf/Twig | | | | | | | | | | | |
| | Extract | | | | | | | | | | | |
| Glycerol 85% | Glycerol | | | | | | | | | | 3.0 | 2.0 |
| Glycerol 99% | Glycerol | 4.0 | 3.0 | | 4.5 | | 3.0 | 4.0 | | | | |
| Hydrolite-5 | 1,2-Pentanediol | | | | | 4.0 | | 5.0 | | | | |
| (Symrise) | | | | | | | | | | | | |
| Isodragol (Symrise) | Triisononanoin | | | | | | | | | | | |
| Isopropyl myristate | Isopropyl | | | | | | | | | | | |
| (Symrise) | Myristate | | | | | | | | | | | |
| Isopropyl palmitate | Isopropyl | | | | | | | | | | 4.0 | |
| (Symrise) | Palmitate | | | | | | | | | | | |
| Karion F (Merck) | Sorbitol | | | | | | | | | | | 2.0 |
| Keltrol RD | Xanthan Gum | | | | | | | | | | 0.2 | |
| (CP-Kelco) | | | | | | | | | | | | |
| Keltrol T ® (Calgon) | Xanthan Gum | | | 0.2 | 0.2 | 0.3 | | | | | | |
| Kojic Acid | Kojic Acid | | | | | | | | | | 1.0 | |
| (Cosmetochem) | | | | | | | | | | | | |
| Lanette E ® (Cognis) | Sodium | | | 0.7 | | | | | | | | |
| | cetearylsulfate | | | | | | | | | | | |
| Lanette O ® (Cognis) | Cetyl and | | 1.1 | | | | | | | 2.5 | | |
| | Stearyl alcohol | | | | | | | | | | | |
| Lanette 16 ® | Cetyl alcohol | | | 1.2 | | | 0.5 | | | | 1.0 | |
| (Cognis) | | | | | | | | | | | | |
| Lanette 18 (Care | Stearyl Alcohol | | | | | | | | | | 4.5 | |
| Chemicals) | | | | | | | | | | | | |
| Lara Care A-200 | Galactoarabinan | | | | | | | 0.2 | | | | |
| (Rahn) | | | | | | | | | | | | |
| Mg Ascorbyl- | Magnesium | | | | | | | | | | 3.0 | |
| phosphate | Ascorbyl- | | | | | | | | | | | |
| | phosphate | | | | | | | | | | | |
| Magnesium Chloride | Magnesium | | | | | | | | | | | 0.7 |
| (Merck) | Chloride | | | | | | | | | | | |
| Monomuls 90-O 18 ® | Glyceryl oleate | 1.0 | | | | | | | | | | |
| (Cognis) | | | | | | | | | | | | |
| Myritol 318 ® | Caprylic/Capric | 6.0 | 5.0 | | | | | | | | | |
| (Cognis) | triglycerides | | | | | | | | | | | |
| NaOH 10% aq. | Sodium | | | 2.8 | | 2.2 | 2.9 | 0.6 | | | | |
| solution | hydroxide | | | | | | | | | | | |
| Sodium Ascorbyl- | Sodium | | | | | | | | | | 2.0 | |
| Phosphate (EMD | Ascorbyl- | | | | | | | | | | | |
| Chemicals) | phosphate | | | | | | | | | | | |
| Natrosol 250 HHR | Hydroxymethyl | | 0.3 | | | | | | | | | |
| (Aqualon) | cellulose | | | | | | | | | | | |
| Neo-Dragocid | Methylparaben, | | | | | | | | | | | |
| powder (Symrise) | Sorbic Acid, | | | | | | | | | | | |
| | Dehydroacetic | | | | | | | | | | | |
| | Acid, | | | | | | | | | | | |
| | Propylparaben | | | | | | | | | | | |
| Neo Heliopan ® AP | Disodium | 10.0 | | 22.0 | | | | | | | | |
| (Symrise), 15% as | phenyl- | | | | | | | | | | | |
| sodium salt | dibenzimidazole | | | | | | | | | | | |
| | tetrasulfonate | | | | | | | | | | | |
| Neo Heliopan AP | Disodium | | | | 22.0 | | | | | | | |
| (Symrise), 10% aq. | phenyl- | | | | | | | | | | | |
| solution neutralized | dibenzimidazole | | | | | | | | | | | |
| with NaOH | tetrasulfonate | | | | | | | | | | | |
| Neo Heliopan ® AV | Ethylhexyl | 4.0 | | | | 5.0 | 6.0 | 2.0 | | | | |
| (Symrise) | methoxycinnamate | | | | | | | | | | | |
| Neo Heliopan ® BB | Benzophenone-3 | | 1.0 | | | | | | | | | |
| (Symrise) | | | | | | | | | | | | |
| Neo Heliopan ® 303 | Octocrylene | | 7.0 | | | | | | | | | |
| (Symrise) | | | | | | | | | | | | |
| Neo Heliopan ® 357 | Butylmethoxy- | | | 2.0 | | 1.5 | 1.5 | 1.5 | 0.5 | 0.5 | | |
| (Symrise) | dibenzoylmethane | | | | | | | | | | | |
| Neo Heliopan ® | Isoamyl p- | 4.0 | | | | 5.0 | | 6.0 | | 2.0 | | |
| E 1000 (Symrise) | methoxy- | | | | | | | | | | | |
| | cinnamate | | | | | | | | | | | |
| Neo Heliopan ® | Homosalate | | | 5.0 | | | | | | | | |
| HMS (Symrise) | | | | | | | | | | | | |
| Neo Heliopan ® | Phenylbenzimidazolesulfonic | | | | 33.3 | 10.0 | 13.3 | | 3.3 | | | |
| Hydro | acid | | | | | | | | | | | |
| (15% aq. solution | | | | | | | | | | | | |
| neutralized with | | | | | | | | | | | | |
| NaOH) (Symrise) | | | | | | | | | | | | |
| Neo Heliopan ® MA | Menthyl | | 3.0 | | | | | | | | | |
| (Symrise) | anthranilate | | | | | | | | | | | |
| Neo Heliopan ® | 4-Methyl- | 2.0 | | | | 2.0 | 4.0 | 3.0 | | | | |
| MBC | benzylidenecamphor | | | | | | | | | | | |
| (Symrise) | | | | | | | | | | | | |
| Neo Heliopan ® OS | Ethylhexyl | 3.0 | | | | | | | | | | |
| (Symrise) | salicylate | | | | | | | | | | | |
| Neutral oil (Symrise) | Caprylic/capric | | | 5.0 | | | 2.0 | | | | 6.0 | |
| | triglycerides | | | | | | | | | | | |
| Octyltriazone | Ethylhexyltriazone | 1.0 | | | | | | | | | | |
| Oxynex 2004 | BHT | | | | | | | | | | | 0.1 |
| (Merck) | | | | | | | | | | | | |
| Paraffin Oil 5 Grade | Paraffinum | | | | | | | | | | | |
| E (Parafluid) | Liquidum | | | | | | | | | | | |
| Perfume Oil | Perfume | 0.3 | 0.3 | 0.3 | | 0.3 | 0.4 | 0.2 | 0.5 | 0.4 | 0.3 | 0.4 |
| (Symrise Fragrance) | (Fragrance) | | | | | | | | | | | |
| PCL Liquid | Cetearyl | | | | | | | | | | | 12.0 |
| (Symrise) | Ethylhexanoate, | | | | | | | | | | | |
| | Isopropyl | | | | | | | | | | | |
| | Myristate | | | | | | | | | | | |
| PCL Liquid 100 | Cetearyl | | | | | | | | | | 3.0 | |
| (Symrise) | Ethylhexanoate | | | | | | | | | | | |
| Pemulen TR 2 | Acrylates/C10-30 | | | | | | | 0.2 | | | | |
| (Novion) | Alkyl | | | | | | | | | | | |
| | Acrylate | | | | | | | | | | | |
| | Crosspolymer | | | | | | | | | | | |
| Permulgin 2550 ® | Bees Wax | 1.0 | | | | | | | | | | |
| (Koster Keunen) | | | | | | | | | | | | |
| Phenoxyethanol | Phenoxyethanol | 0.7 | | 0.7 | | 0.7 | 0.7 | 0.7 | | | | |
| (Symrise) | | | | | | | | | | | | |
| Polymer JR 400 | Polyquaternium- | | | | | | | | 0.4 | | | |
| | 10 | | | | | | | | | | | |
| 1,2-Propylene glycol | Propylene | | | | | | | | | | | |
| | Glycol | | | | | | | | | | | |
| Retinyl Palmitate in | Retinyl | | | | | | | | | | | 0.2 |
| Oil (DSM Nutritional | Palmitate | | | | | | | | | | | |
| Products) | | | | | | | | | | | | |
| Softigen 767 | PEG-6 | | | | | | | | 2.5 | | | |
| | Caprylic/Capric | | | | | | | | | | | |
| | Glycerides | | | | | | | | | | | |
| Solubilizer (Symrise) | PEG 40 | | | | | | | | 3.0 | | | |
| | Hydrogenated | | | | | | | | | | | |
| | Castor Oil, | | | | | | | | | | | |
| | Trideceth-9, | | | | | | | | | | | |
| | Propylene | | | | | | | | | | | |
| | glycol, Water | | | | | | | | | | | |
| Sun Flower Oil | Helianthus | | | | | | | | | | | 5.0 |
| (Wagner) | Annuus | | | | | | | | | | | |
| | (Sunflower) | | | | | | | | | | | |
| | Seed Oil | | | | | | | | | | | |
| Sweet Almond Oil | Prunus dulcis | | | | | | | | | | | 5.0 |
| (Wagner) | | | | | | | | | | | | |
| SymCalmin | Butylene Glycol, | | | 0.5 | | | | | | | | |
| | Pentylene | | | | | | | | | | | |
| | Glycol, | | | | | | | | | | | |
| | Hydroxyphenyl | | | | | | | | | | | |
| | Propamidobenzoic | | | | | | | | | | | |
| | Acid | | | | | | | | | | | |
| Symdiol 68 | 1,2-Hexanediol, | | | 0.5 | | | | | | | 0.5 | |
| (Symrise) | Caprylyl glycol | | | | | | | | | | | |
| SymMatrix (Symrise) | Maltodextrin, | | | | | | | | | | | 1.0 |
| | Rubus | | | | | | | | | | | |
| | Fruticosus | | | | | | | | | | | |
| | (Blackberry) | | | | | | | | | | | |
| | Leaf Extract | | | | | | | | | | | |
| SymWhite 377 | 4-(1- | | | | | | | | | | 0.5 | |
| | Phenylethyl)- | | | | | | | | | | | |
| | 1,3-benzenediol | | | | | | | | | | | |
| Tegosoft TN ® | C12-C15 Alkyl | 6.0 | | | | 4.0 | 2.0 | | | | | |
| (Goldschmidt) | benzoates | | | | | | | | | | | |
| Texapon N 70 | Sodium Laureth | | | | | | 0.1 | 0.5 | | | | |
| (Cognis) | Sulfate | | | | | | | | | | | |
| Texapon NSO BZ | Sodium Laureth | | | | | | | | 27.0 | | | |
| (Cognis) | Sulfate | | | | | | | | | | | |
| Titanium dioxide | Titanium dioxide | | 5.0 | | | | | | | | | |
| microfine | | | | | | | | | | | | |
| Tocopherol Acetate | Tocopheryl | | | | | | | | | | | 3.0 |
| (DSM Nutritional | Acetate | | | | | | | | | | | |
| Products) | | | | | | | | | | | | |
| Unimer U-151 | PVP/Hexadecene | | | | | 0.5 | | | | | | |
| (Induchem) | Copolymer | | | | | | | | | | | |
| Veegum ultra ® | Magnesium | | 1.0 | | | | | | | | | |
| (Vanderbilt) | Aluminium | | | | | | | | | | | |
| | sulfate | | | | | | | | | | | |
| Witch Hazel | Hamamelis | | | | | | | | 1.0 | | | |
| Distillate (Symrise) | Virginiana | | | | | | | | | | | |
| | (Witch Hazel) | | | | | | | | | | | |
| Zinc oxide neutral | Zinc oxide | 7.0 | | | | | | | | | | |
| (Symrise) | | | | | | | | | | | | |
| Water, dist. | Aqua (Water) | ad | ad | ad | ad | ad | ad | ad | ad | ad | ad | ad |
| | | 100 | 100 | 100 | 100 | 100 | 100 | 100 | 100 | 100 | 100 | 100 |

### Specific Embodiments

Specific embodiment one comprises a compound of formula (III) or (V):

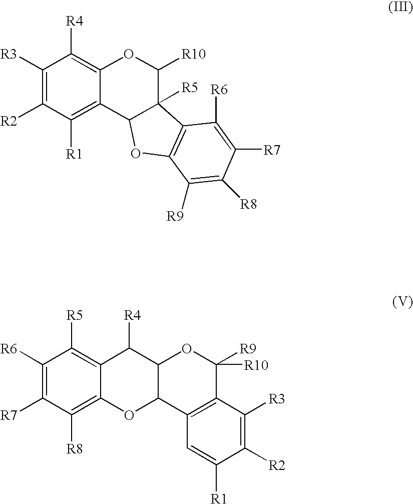

in which
R¹ to R¹⁰ independently of one another can be hydrogen, hydroxyl, C₁-C₁₀-alkyl, C₁-C₁₀-alkenyl, C₁-C₁₀-alkoxy, prenyl or O-glycosyl, and two radicals R¹ to R¹⁰ can be joined via a methylenedioxy group —O—CH₂—O—, and
R⁵ can be replaced by a double bond, as a drug.

Specific embodiment two comprises a drug according to specific embodiment one wherein the compound is selected from the group comprising medicarpin, maackiain, variabilin, anhydrovariabilin and peltogynol.

Specific embodiment three comprises a drug according to specific embodiment one which is intended for

(a) reducing or preventing a translocation of the AhR into a cell nucleus,
(b) reducing or preventing a UVB-induced or UVB-inducible gene expression,
(c) reducing or preventing a gene expression induced or inducible by polycyclic aromatic hydrocarbons, preferably TCDD, and/or
(d) reducing or preventing UVB-induced or UVB-inducible skin damage, especially skin cancer, skin ageing, skin inflammations and sunburn. Specific embodiment four comprises a cosmetic or pharmaceutical formulation consisting of, consisting essentially of or comprising a compound of formula (III) or (V), as defined in specific embodiment one, in a sufficient amount for
(a) reducing or preventing a translocation of the AhR into a cell nucleus,
(b) reducing or preventing a UVB-induced or UVB-inducible gene expression,
(c) reducing or preventing a gene expression induced or inducible by polycyclic aromatic hydrocarbons, preferably TCDD, and/or
(d) reducing or preventing UVB-induced or UVB-inducible skin damage, especially skin cancer, skin ageing, skin inflammations and sunburn, and a UV filter.

Specific embodiment five comprises a formulation according to specific embodiment four containing the compound in a proportion of at least 0.0001 wt. % and preferably in a proportion of 0.0005 to 15 wt. %, based in each case on the total formulation.

Specific embodiment six comprises a formulation according to specific embodiments four or five, characterized in that it is a cosmetic formulation preferably selected from the group comprising a sun cream, a skin protection lotion and an after-sun lotion.

Specific embodiment seven comprises a process for the preparation of a drug according to one of specific embodiments one to three or a formulation according to one of specific embodiments four to six, comprising the extraction of wood for up to 72 hours with an extractant which comprises water, ethyl acetate, an alcohol and/or a ketone selected from the group comprising methanol, ethanol, n-propanol, isopropanol and acetone, and mixtures of two or more of these substances.

Specific embodiment eight comprises a process according to specific embodiment seven wherein the wood is selected from the wood of Acacia species, Albizzia procera, Alysicarpus sp., Amorpha californica, Andira inermis, Apios tuberosa, Artemisia indica, Astragalus species, Baphia nitida, Baptisia tinctoria, Berchemia species, Bituminaria species, Bolusanthus speciosus, Bowdichia nitida, Brya ebenus, Caesalpinia species, Calopogonium mucunoides, Cassine species, Ceratostigma minus, Cicer species, Cladrastis platycarpa, Colophospermum mopane, Crotalaria species, Dalbergia species, Dalea filiciformis, Derris species, Desmodium gangeticum, Distemonanthus benthamianus, Dolichos biflorus, Elaeodendron balae, Entandrophragma cylindricum, Erythrina species, Euchresta horsfieldii, Flemingia chappar, Glycine species, Glycyrrhiza species, Goniorrhachis marginata, Harpalyce brasiliana, Hedysarum multijugum, Iris bungei, Lablab niger, Leguminosae, especially of the subfamily Papilionoideae, Lespedeza species, Lonchocarpus species, Maackia species, Machaerium species, Medicago sativa, Melilotus species, Millettia species, Mundulea striata, Myroxylon peruiferum, Neorautanenia species, Nissolia fruticosa, Ononis vaginalis, Oroxylum indicum, Osteophloeum platyspermum, Pachyrrhizus species, Peltogyne species, Pericopsis species, Petalostemon purpureus, Phaseolus species, Pisum sativum, Platymiscium trinitatis, Psophocarpus tetragonolobus, Pterocarpus species, Pueraria species, Sophora species, Spatholobus suberectus, Swartzia species, Tephrosia species, Trachylobium species, Trifolium species, Ulex species, Umtiza listeriana, Vigna unguiculata, Virgilia oroboides, Woodsia scopulina, or mixtures of two or more of these woods.

Specific embodiment nine comprises a process according to specific embodiments seven or eight, characterized in that the extraction is carried out for at least 1 hour, preferably for at least 2 hours and particularly preferably for at most 24 hours.

Specific embodiment ten comprises a use of an extract which can be prepared by the process of one of specific embodiments seven to nine for the preparation of a drug or a cosmetic or pharmaceutical formulation for

(a) reducing or preventing a translocation of the AhR into a cell nucleus,
(b) reducing or preventing a UVB-induced or UVB-inducible gene expression,
(c) reducing or preventing a gene expression induced or inducible by polycyclic aromatic hydrocarbons, preferably TCDD, and/or
(d) reducing or preventing UVB-induced or UVB-inducible skin damage, especially skin cancer, skin ageing, skin inflammations and sunburn.

1. A compound of formula (III) or (V): 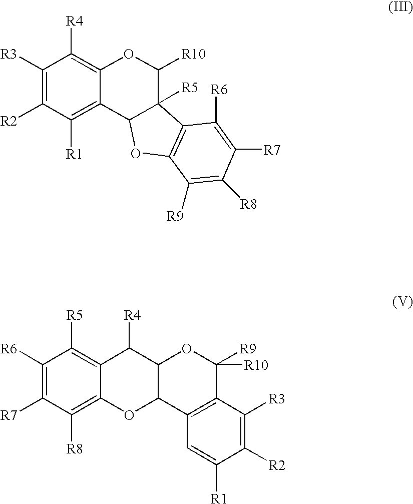
in which
R¹ to R¹⁰ independently of one another is hydrogen, hydroxyl, C₁-C₁₀-alkyl, C₁-C₁₀-alkenyl, C₁-C₁₀-alkoxy, prenyl or O-glycosyl, and two radicals R¹ to R¹⁰ can be joined via a methylenedioxy group —O—CH₂—O—, and
R⁵ can be replaced by a double bond,
wherein the compound is suitable for use as a drug.

2. The compound according to claim 1 wherein the compound is selected from the group comprising medicarpin, maackiain, variabilin, anhydrovariabilin and peltogynol.

3. The compound according to claim 1 which is intended for
(a) reducing or preventing a translocation of the AhR into a cell nucleus,
(b) reducing or preventing a UVB-induced or UVB-inducible gene expression,
(c) reducing or preventing a gene expression induced or inducible by polycyclic aromatic hydrocarbons, preferably TCDD, and/or
(d) reducing or preventing UVB-induced or UVB-inducible skin damage, especially skin cancer, skin ageing, skin inflammations and sunburn.

4. A cosmetic or pharmaceutical formulation consisting of, consisting essentially of or comprising a compound of formula (III) or (V), according to claim 1, in a sufficient amount for
(a) reducing or preventing a translocation of the AhR into a cell nucleus,
(b) reducing or preventing a UVB-induced or UVB-inducible gene expression,
(c) reducing or preventing a gene expression induced or inducible by polycyclic aromatic hydrocarbons and/or
(d) reducing or preventing UVB-induced or UVB-inducible skin damage and a UV filter.

5. The formulation according to claim 4 wherein the compound is in a proportion of at least 0.0001 wt. % based in each case on the total formulation.

6. The formulation according to claim 4, wherein the formulation is a cosmetic formulation.

7. A process for the preparation of a compound according to claim 1 or a formulation
comprising a compound of formula (III) or (V), according to claim 1, in a sufficient amount for
(a) reducing or preventing a translocation of the AhR into a cell nucleus,
(b) reducing or preventing a UVB-induced or UVB-inducible gene expression,
(c) reducing or preventing a gene expression induced or inducible by polycyclic aromatic hydrocarbons and/or
(d) reducing or preventing UVB-induced or UVB-inducible skin damage,
comprising the extraction of wood for up to 72 hours with an extractant which comprises water, ethyl acetate, an alcohol and/or a ketone selected from the group comprising methanol, ethanol, n-propanol, isopropanol and acetone, and mixtures of two or more of these substances.

8. A process according to claim 7 wherein the wood is selected from the wood of Acacia species, Albizzia procera, Alysicarpus sp., Amorpha californica, Andira inermis, Apios tuberosa, Artemisia indica, Astragalus species, Baphia nitida, Baptisia tinctoria, Berchemia species, Bituminaria species, Bolusanthus speciosus, Bowdichia nitida, Brya ebenus, Caesalpinia species, Calopogonium mucunoides, Cassine species, Ceratostigma minus, Cicer species, Cladrastis platycarpa, Colophospermum mopane, Crotalaria species, Dalbergia species, Dalea filiciformis, Derris species, Desmodium gangeticum, Distemonanthus benthamianus, Dolichos biflorus, Elaeodendron balae, Entandrophragma cylindricum, Erythrina species, Euchresta horsfieldii, Flemingia chappar, Glycine species, Glycyrrhiza species, Goniorrhachis marginata, Harpalyce brasiliana, Hedysarum multijugum, Iris bungei, Lablab niger, Leguminosae, Leguminosae subfamily Papilionoideae, Lespedeza species, Lonchocarpus species, Maackia species, Machaerium species, Medicago sativa, Melilotus species, Millettia species, Mundulea striata, Myroxylon peruiferum, Neorautanenia species, Nissolia fruticosa, Ononis vaginalis, Oroxylum indicum, Osteophloeum platyspermum, Pachyrrhizus species, Peltogyne species, Pericopsis species, Petalostemon purpureus, Phaseolus species, Pisum sativum, Platymiscium trinitatis, Psophocarpus tetragonolobus, Pterocarpus species, Pueraria species, Sophora species, Spatholobus suberectus, Swartzia species, Tephrosia species, Trachylobium species, Trifolium species, Ulex species, Umtiza listeriana, Vigna unguiculata, Virgilia oroboides, Woodsia scopulina, or mixtures of two or more of these woods.

9. A process according to claim 7, wherein the extraction is carried out for at least 1 hour.

10. A method for making an extract which can be prepared by the process of claim 7 for the preparation of a drug or a cosmetic or pharmaceutical formulation for
(a) reducing or preventing a translocation of the AhR into a cell nucleus,
(b) reducing or preventing a UVB-induced or UVB-inducible gene expression,
(c) reducing or preventing a gene expression induced or inducible by polycyclic aromatic hydrocarbons and/or
(d) reducing or preventing UVB-induced or UVB-inducible skin damage.

11. The formulation of claim 4, wherein the polycyclic aromatic hydrocarbon is 2,3,7,8-tetrachlorodibenzo-p-dioxin (TCDD).

12. The formulation of claim 4, wherein the skin damage is skin cancer, skin ageing, skin inflammation, or sunburn.

13. The formulation of claim 5, wherein the compound is in a proportion of 0.0005 wt % to 15 wt %.

14. The formulation of claim 6, wherein the formulation is a sun cream, a skin protection lotion, or an after-sun lotion.

15. The process of claim 9, wherein the extraction is carried out for at least 2 hours.

16. The process of claim 9, wherein the extraction is carried out for at most 24 hours.

17. The method of claim 10, wherein the polycyclic aromatic hydrocarbon is TCDD.

18. The method of claim 10, wherein the skin damage is skin cancer, skin ageing, skin inflammation, or sunburn.
